(19) United States
(12) Patent Application Publication
(10) Pub. No.: US 2021/0284840 A1
(43) Pub. Date: Sep. 16, 2021

(54) COPOLYMER COMPOSITION FOR COATING AND ADHESIVE APPLICATIONS

(71) Applicant: Dow Silicones Corporation, Midland, MI (US)

(72) Inventors: Bizhong Zhu, Midland, MI (US); Martin Grasmann, Midland, MI (US); Vinita Pandit, Midland, MI (US)

(21) Appl. No.: 16/334,562

(22) PCT Filed: Aug. 18, 2017
(86) PCT No.: PCT/US2017/047468

§ 371 Date: Mar. 19, 2019

Related U.S. Application Data
(60) Provisional application No. 62/396,336, filed on Sep. 19, 2016.

(51) Int. Cl. C08L 83/04; C09J 183/04; C09J 175/06; C09D 183/04; C09D 175/06; C08L 75/06; A61K 9/00; A61K 45/06; A61K 33/38; A61K 31/616; A61K 31/19; A61K 31/192; A61K 31/405; A61K 31/167; A61K 31/245; A61K 31/045; A61K 47/34; A61L 24/04

(52) U.S. Cl. CPC C08L 83/04; A61F 13/0253; C09J 175/06; C09D 183/04; C09D 175/06; C08L 75/06; A61K 9/0014; A61K 45/06; A61K 33/38; A61K 31/616; A61K 31/19; A61K 31/192; A61K 31/405; A61K 31/167; A61K 31/245; A61K 31/045; A61K 47/34; A61L 24/043; C09J 183/04

(57) ABSTRACT

An organic-siloxane copolymer composition is useful for preparing a skin contact adhesive or a coating. A skin contact adhesive composition includes (I) an organic-siloxane copolymer composition and (II) an excipient. A skin contact adhesive prepared by hardening the composition is useful in applications such as adhesives for medical tapes, adhesives for wound dressings, adhesives for prosthetics, ostomy appliance adhesives, adhesives for medical monitoring appliances, adhesives for scar therapy treatments, adhesives for cosmetic patches, and transdermal drug delivery systems. A coating composition includes (a) the organic-siloxane copolymer composition and (b) a coating additive. The coating prepared by hardening the coating composition is useful for treating leather.

### CROSS REFERENCE TO RELATED APPLICATIONS

This application claims priority to U.S. Provisional Patent Application Ser. No. 62/396,336 filed on 19 Sep. 2016. U.S. Provisional Patent Application Ser. No. 62/396,336 is hereby incorporated by reference.

### BACKGROUND

### Technical Field

A copolymer composition is useful for preparing a skin contact adhesive and/or a coating on a substrate. Methods for the preparation and use of the copolymer composition are disclosed. The copolymer composition includes an organic-siloxane copolymer.

### Introduction

Various types of skin contact adhesives have been proposed for skin contact applications such as adhesives for medical tapes, adhesives for wound dressings, adhesives for prosthetics, ostomy appliance adhesives, adhesives for medical monitoring appliances, adhesives for scar therapy treatments, and transdermal drug delivery systems. Hydrocolloid adhesives and acrylate adhesives typically have the highest adhesion (e.g., require the highest energy to remove from the skin). Polyurethane adhesives have the next highest adhesion, and silicones have the lowest adhesion of these types of skin contact adhesives. Those skin contact adhesives with higher adhesion (requiring higher energy to remove) can cause more pain and potential trauma to the skin during removal than those with lower energy required for removal. Certain skin contact adhesives may also leave an undesirable residue on skin during removal.

In the process of chronic wound care, adhesive wound dressings and/or medical tapes, may cause pain and injury in and around the wound during dressing changes. Repeated application and removal of skin contact adhesives can be painful and traumatic, especially for patients with fragile skin. Fragile skin is generally characterized by thin skin that tears easily and may be more common in older adults than other populations. Aging, sun exposure, and genetics all play a role in thinning of the skin. Certain medications, such as long-term use of oral or topical corticosteroids, can also weaken skin and the blood vessels within the skin and make it more vulnerable to trauma associated with removal of adhesives. Individuals with fragile skin can also experience a loss of cohesion between the epidermis and dermis and between the dermis and subcutaneous tissue, making these individuals more prone to skin tears and trauma, particularly when skin contact adhesives with higher adhesion are used.

Furthermore, silicone adhesives, e.g., those prepared from two part catalyzed silicone elastomers, may be unsuitable for use in certain skin contact adhesive applications, such as transdermal drug delivery. Certain catalysts used to prepare silicone elastomers (such as platinum group metal catalysts for hydrosilylation) may detrimentally affect the medically active ingredient in transdermal drug delivery devices.

In addition to skin contact adhesives, polyurethanes and polyorganosiloxanes are also used for coatings applied on various substrates. Polyurethanes are known to have high mechanical toughness but have limitations such as limited temperature resistance, moisture resistance, and radiation stability. Polyorganosiloxanes are environmentally very stable.

Incorporating some polyorganosiloxane into a polyurethane based coating is challenging in the industry because the chemical natures of polyorganosiloxanes and polyurethanes have very limited compatibility.

### Problem to be Solved

There is an industry need to develop a composition that can be used to form a skin contact adhesive with one or more of the following benefits: good adhesive properties, ability to transfer an active ingredient e.g., in transdermal drug delivery applications, moisture resistance (from the environment to the skin), water transport from the skin to the environment, stability, minimal skin irritation, minimal damage to the skin during use and removal, and/or minimal residue on skin during and after removal. There is also an industry need to develop a composition that can be used to form a coating on a substrate with one or more of the following benefits: improved compatibility between polyurethane and silicones, improved weathering resistance, hydrophobicity, hydrolytic stability, radiation resistance, thermal resistance, corrosion resistance, surface smoothness and gloss, scratch resistance, lower viscosity at similar solid content (impacting volatile organic content, VOC), and reduced friction.

### SUMMARY OF THE INVENTION

A copolymer composition comprises two or more starting materials. The copolymer composition comprises at least one of copolymer (A) and copolymer (B), where copolymer (A) is an organic-siloxane copolymer comprising units of formulae:

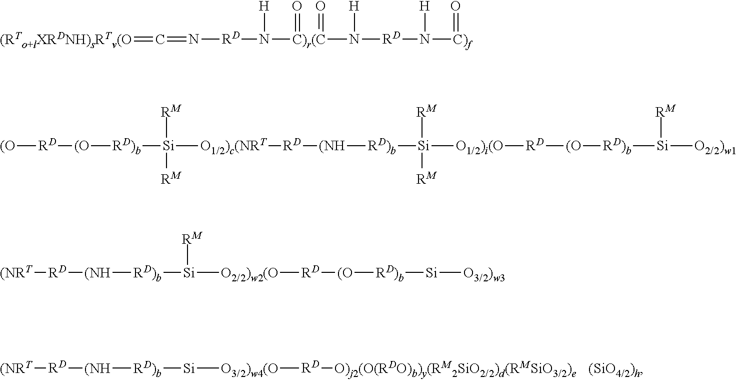

where
each R^{D} is independently a divalent hydrocarbon group or a divalent halogenated hydrocarbon group;
each R^{M} is independently a monovalent hydrocarbon group or a monovalent halogenated hydrocarbon group;
each R^{T} is independently hydrogen or a hydrocarbon group;
each subscript b is independently 0 to 1,000,000;
subscript c is 0 to 200,000, subscript i is 0 to 200,000, subscript w1 is 0 to 200,000, subscript w2 is 0 to 200,000, subscript w3 is 0 to 200,000, subscript w4 is 0 to 200,000, and a quantity (c+i+w1+w2+w3+w4) is ≥1;
subscript d is 0 to 1,000,000;
subscript e is 0 to 1,000,000;
subscript f is 0 to 1,500,000;
subscript h is ≥0;
subscript j1 is ≥0;
each X is independently nitrogen, oxygen, or sulfur;
subscript o=0 when X is oxygen or sulfur, and subscript o=1 when X is nitrogen;
subscript r is 0 to 1,500,000, and the quantity f+r is ≥1;
subscript s is 0 to 200,000; and
subscript v is 0 to 200,000;
subscript y is ≥0;
and copolymer (B) is an organic-siloxane copolymer comprising units of formulae:

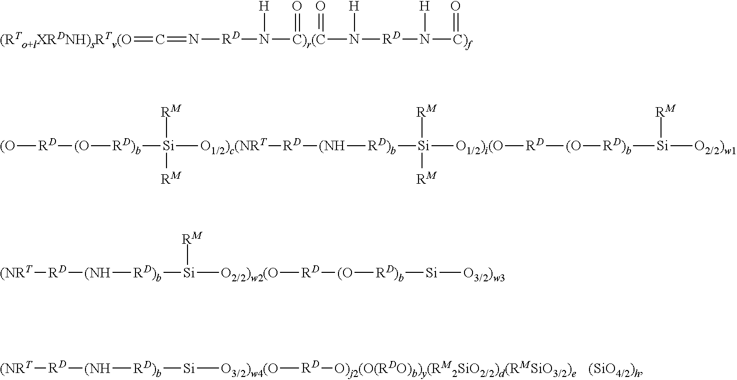

where R^{T}, R^{D}, R^{M}, and subscripts o, l, s, v, r, c, l, w1, w2, w3, w4, b, and y are as defined above for copolymer (A) and subscript j2 is >0, and if j1 is >0, then j2/j1 is >1.1. Copolymer (A) and copolymer (B) are distinct from one another. The blend may further comprise one or both of
(C) an organic polyol; or
(D) a reaction product of an organic polyisocyanate and an organic polyol.

A skin contact adhesive composition comprises the copolymer composition described above, and the skin contact adhesive composition hardens to form a skin contact adhesive. The skin contact adhesive is useful in various skin contact adhesive applications, including adhesives for medical tapes, adhesives for wound dressings, adhesives for prosthetics, ostomy appliance adhesives, adhesives for medical monitoring appliances, adhesives for scar therapy treatments, adhesives for cosmetic patches, and transdermal drug delivery systems.

A coating composition comprises the copolymer composition described above. The coating composition can be applied onto various substrates and can be hardened to form a coating on the substrate.

### BRIEF DESCRIPTION OF THE DRAWINGS

FIG. 1 is a partial cross section of a laminate article 100 including the skin contact adhesive described herein.

FIG. 2A shows a perspective view of a wound dressing in the form of an adhesive bandage 200 including the skin contact adhesive 202 described herein.

FIG. 3 is a partial cross section of a wound dressing in the form of a laminate article 300 including the skin contact adhesive 308 described herein.

FIG. 4 shows a flange 400 for use in an ostomy appliance including the skin contact adhesive 402 described herein.

Reference Numerals

100 laminate article
101 support
102 skin contact adhesive
103 release liner
104 skin facing surface
105 skin contacting surface
200 adhesive bandage
201 absorbent layer
202 skin contact adhesive
203 skin facing surface
204 support
205 skin contact surface
300 laminate article
301 opposed surface of the carrier
302 carrier
303 opposed surface of the support
304 support
305 skin facing surface of the support
306 absorbent layer
307 opposed surface of the skin contact adhesive
308 skin contact adhesive
309 skin facing surface of the skin contact adhesive
310 release liner
400 flange
401 support member
402 skin contact adhesive
403 aperture

### DETAILED DESCRIPTION OF THE INVENTION

A copolymer composition comprises at least one of copolymer (A) and copolymer (B). The copolymer composition may optionally further comprise one or both of starting material (C) an organic polyol; and starting material (D) a reaction product of an organic polyisocyanate and an organic polyol. The copolymer composition comprises at least two starting materials. The copolymer composition may comprise (A) and (B). Alternatively, the copolymer composition may comprise (A) and (C). Alternatively, the copolymer composition may comprise (B) and (C). Alternatively, the copolymer composition may comprise (A) and (D). Alternatively, the copolymer composition may comprise (B) and (D). Alternatively, the copolymer composition may comprise (A), (B), and (C). Alternatively, the copolymer composition may comprise (A), (B), and (D). Alternatively, the copolymer composition may comprise (A), (C), and (D). Alternatively, the copolymer composition may comprise (B), (C), and (D). Alternatively, the copolymer composition may comprise (A), (B), (C), and (D).

### Copolymer (A)

Copolymer (A) is an organic-siloxane copolymer. Copolymer (A) comprises units of formulae:

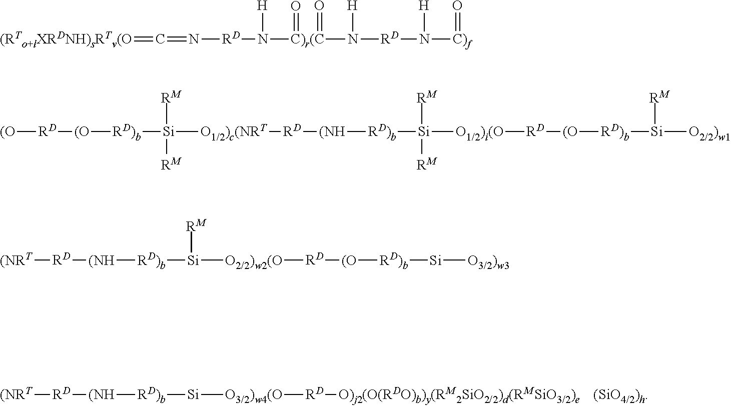

In the unit formula above, each R^{D} is independently a divalent hydrocarbon group or a divalent halogenated hydrocarbon group, as defined below. Each R^{D} may independently have 2 to 13 carbon atoms. Alternatively, each R^{D} may be selected from alkylene such as ethylene or propylene, arylene such as phenylene, or alkaralkylene. Alternatively, each R^{D} may be an alkylene group such as ethylene or propylene.

Each R^{M} is independently a monovalent hydrocarbon group or a monovalent halogenated hydrocarbon group as defined below. Each R^{M} may have 1 to 13 carbon atoms. Alternatively, each R^{M} may be a monovalent hydrocarbon group free of aliphatic unsaturation. For example, each R^{M} may be independently selected from alkyl such as methyl, ethyl, propyl, butyl or hexyl; aryl such as phenyl, or aralkyl such as tolyl, xylyl or phenyl-methyl. Alternatively, each R^{M} may be methyl or phenyl, and alternatively each R^{M} may be methyl.

Each R^{T} is hydrogen or a monovalent hydrocarbon group. The monovalent hydrocarbon group for R^{T} may have 1 to 13 carbon atoms. The monovalent hydrocarbon group for R^{T} is group independently selected from alkyl such as methyl, ethyl, propyl, butyl, or hexyl; aryl such as phenyl; or aralkyl such as tolyl, xylyl, or phenyl-methyl. Alternatively, each R^{T} may be methyl or phenyl. Alternatively each R^{T} may be hydrogen or methyl.

Each subscript b is independently greater than or equal to 0. Each instance of subscript b can have a different value in a different unit of the copolymer. Alternatively, subscript b is 0 to 1,000,000. Alternatively, subscript b is 0 to 200,000. Alternatively, subscript b is 0 to 100,000. Alternatively, subscript b is 0 to 50,000. Alternatively, subscript b is 0 to 10,000. Alternatively, subscript b is 0 to 5,000. Alternatively, subscript b is 0 to 1,000. Alternatively, subscript b is 0 to 500. Alternatively, subscript b is 0 to 100. Alternatively, subscript b is 1 to 100. Alternatively, subscript b is 1 to 50. Alternatively, subscript b is 1 to 20. Alternatively, subscript b is 0 to 1. Alternatively, subscript b=1. Alternatively, subscript b=2. Alternatively, subscript b=3. Alternatively, subscript b=4. Alternatively, subscript b=5.

Subscript c≥0. Alternatively, subscript c is 0 to 200,000. Alternatively, subscript c is 0 to 100,000. Alternatively, subscript c is 0 to 50,000. Alternatively, subscript c is 0 to 10,000. Alternatively, subscript c is 0 to 5,000. Alternatively, subscript c is 0 to 1,000. Alternatively, subscript c is 0 to 500. Alternatively, subscript c is 0 to 100. Alternatively, subscript c is 0 to 50. Alternatively, subscript c is 0 to 20. Alternatively, subscript c is 0 to 10. Alternatively, subscript c is 1 to 100. Alternatively, subscript c is 1 to 50. Alternatively, subscript c is 1 to 20. Alternatively, subscript c is 1 to 10.

Subscript i≥0. Alternatively, subscript i is 0 to 200,000. Alternatively, subscript i is 0 to 100,000. Alternatively, subscript i is 0 to 50,000. Alternatively, subscript i is 0 to 10,000. Alternatively, subscript i is 0 to 5,000. Alternatively, subscript i is 0 to 1,000. Alternatively, subscript i is 0 to 500. Alternatively, subscript i is 0 to 100. Alternatively, subscript i is 0 to 50. Alternatively, subscript i is 0 to 20. Alternatively, subscript i is 0 to 10. Alternatively, subscript i is 1 to 100. Alternatively, subscript i is 1 to 50. Alternatively, subscript i is 1 to 20. Alternatively, subscript i is 1 to 10.

Subscript w1≥0. Alternatively, subscript w1 is 0 to 200,000. Alternatively, subscript w1 is 0 to 50,000. Alternatively, subscript w1 is 0 to 10,000. Alternatively, subscript w1 is 0 to 5,000. Alternatively, subscript w1 is 0 to 1,000. Alternatively, subscript w1 is 0 to 500. Alternatively, subscript w1 is 0 to 100. Alternatively, subscript w1 is 0 to 50. Alternatively, subscript w1 is 0 to 20. Alternatively, subscript w1 is 0 to 10. Alternatively, subscript w1 is 1 to 100. Alternatively, subscript w1 is 1 to 50. Alternatively, subscript w1 is 1 to 20. Alternatively, subscript w1 is 1 to 10.

Subscript w2≥0. Alternatively, subscript w2 is 0 to 200,000. Alternatively, subscript w2 is 0 to 50,000. Alternatively, subscript w2 is 0 to 10,000. Alternatively, subscript w2 is 0 to 5,000. Alternatively, subscript w2 is 0 to 1,000. Alternatively, subscript w2 is 0 to 500. Alternatively, subscript w2 is 0 to 100. Alternatively, subscript w2 is 0 to 50. Alternatively, subscript w2 is 0 to 20. Alternatively, subscript w2 is 0 to 10. Alternatively, subscript w2 is 1 to 100. Alternatively, subscript w2 is 1 to 50. Alternatively, subscript w2 is 1 to 20. Alternatively, subscript w2 is 1 to 10.

Subscript w3≥0. Alternatively, subscript w3 is 0 to 200,000. Alternatively, subscript w3 is 0 to 50,000. Alternatively, subscript w3 is 0 to 10,000. Alternatively, subscript w3 is 0 to 5,000. Alternatively, subscript w3 is 0 to 1,000. Alternatively, subscript w3 is 0 to 500. Alternatively, subscript w3 is 0 to 100. Alternatively, subscript w3 is 0 to 50. Alternatively, subscript w3 is 0 to 20. Alternatively, subscript w3 is 0 to 10. Alternatively, subscript w3 is 1 to 100. Alternatively, subscript w3 is 1 to 50. Alternatively, subscript w3 is 1 to 20. Alternatively, subscript w3 is 1 to 10.

Subscript w4≥0. Alternatively, subscript w4 is 0 To 200,000. Alternatively, subscript w4 is 0 to 50,000. Alternatively, subscript w4 is 0 to 10,000. Alternatively, subscript w4 is 0 to 5,000. Alternatively, subscript w4 is 0 to 1,000. Alternatively, subscript w4 is 0 to 500. Alternatively, subscript w4 is 0 to 100. Alternatively, subscript w4 is 0 to 50. Alternatively, subscript w4 is 0 to 20. Alternatively, subscript w4 is 0 to 10. Alternatively, subscript w4 is 1 to 100. Alternatively, subscript w4 is 1 to 50. Alternatively, subscript w4 is 1 to 20. Alternatively, subscript w4 is 1 to 10.

A quantity (c+i+w1+w2+w3+w4)≥1. Alternatively, in one embodiment i=w2=w4=0, and a quantity (c+w1+w3)≥1, for example, when the copolymer is prepared using a carbinol-functional polyorganosiloxane, as described below. In an alternative embodiment, c=w1=w3=0, and a quantity (i+w1+w3)≥1, for example, when the copolymer is prepared using an amine-functional polyorganosiloxane, as described below.

Each X is independently nitrogen (N), oxygen (O), or sulfur (S). Alternatively, X is N or O. Alternatively, each X is N. Alternatively, each X is O. Subscript o=0 when X is O or S, and subscript o=1 when X is N.

Subscripts d, e, and h depend on the molecular weight of one of the siloxane segments in the copolymer and may be without limit (e.g., bound only by the molecular weights reachable by the state of the art of siloxane synthesis chemistry). However, subscript d may be 0 to 1,000,000; subscript e may be 0 to 1,000,000; subscript h may be 0 to 1,000,000, and with the proviso that a quantity (d+e+h)≥1. Subscript d≥0. Alternatively, subscript d>0. Alternatively, subscript d is 0 to 200,000, alternatively 0 to 100,000, alternatively 0 to 50,000, alternatively 0 to 10,000, alternatively 0 to 5,000, alternatively 0 to 1,000, alternatively 1 to 1,000, alternatively 1 to 500, and alternatively 1 to 200.

Subscript e≥0. Alternatively, subscript e is 0 to 1,000,000. Alternatively, subscript e is 0 to 200,000, and alternatively 0 to 100,000, alternatively 0 to 50,000, alternatively 0 to 10,000, alternatively 0 to 5,000, alternatively 0 to 1,000, alternatively 1 to 1,000, alternatively 1 to 500, and alternatively 1 to 200. Alternatively, subscript e=0.

Subscript f indicates the number of urethane and/or urea units in the copolymer. Subscript f≥0. Alternatively, subscript f is 0 to 1,500,000. Alternatively, subscript f is 1 to 500,000, and alternatively 1 to 200,000, alternatively 1 to 50,000, alternatively 1 to 10,000, alternatively 1 to 5,000, alternatively 1 to 1,000, alternatively 1 to 500, and alternatively 1 to 200.

Subscript h is ≥0. Alternatively, subscript h is 0 to 1,000,000. Alternatively, subscript h is 0 to 200,000, and alternatively 0 to 100,000, alternatively 0 to 50,000, alternatively 0 to 10,000, alternatively 0 to 5,000, alternatively 0 to 1,000, alternatively 1 to 1,000, alternatively 1 to 500, and alternatively 1 to 200. Alternatively, subscript h=0.

Subscript j1 is ≥0. Alternatively, subscript j1 is >0 to 500,000. Alternatively, subscript j1 is >0 to 200,000, and alternatively 20 to 100,000, alternatively 50 to 50,000, alternatively 100 to 10,000, alternatively 1,000 to 5,000, alternatively 100 to 1,000, alternatively 10 to 500, and alternatively 15 to 200.

Subscript s is ≥0. Alternatively, subscript s is 0 to 200,000. Alternatively, subscript s is 0 to 150,000, and alternatively 0 to 100,000, alternatively 0 to 50,000, alternatively 1 to 10,000, alternatively 1 to 5,000, alternatively 1 to 1,000, alternatively 1 to 500, and alternatively 1 to 200.

Subscript v is ≥0. Alternatively, subscript v is 0 to 200,000. Alternatively, subscript v is 0 to 150,000, and alternatively 0 to 100,000, alternatively 0 to 50,000, alternatively 1 to 10,000, alternatively 1 to 5,000, alternatively 1 to 1,000, alternatively 1 to 500, and alternatively 1 to 200.

Subscript y is ≥0. Alternatively, subscript y is 0 to 200,000. Alternatively, subscript y is 0 to 150,000, and alternatively 0 to 100,000, alternatively 0 to 50,000, alternatively 1 to 10,000, alternatively 1 to 5,000, alternatively 1 to 1,000, alternatively 1 to 500, alternatively 1 to 200, alternatively 1 to 20, and alternatively 1.

Alternatively, when subscripts c=i=w2=w3=w4=e=h=0, copolymer (A) may have unit formula (I):

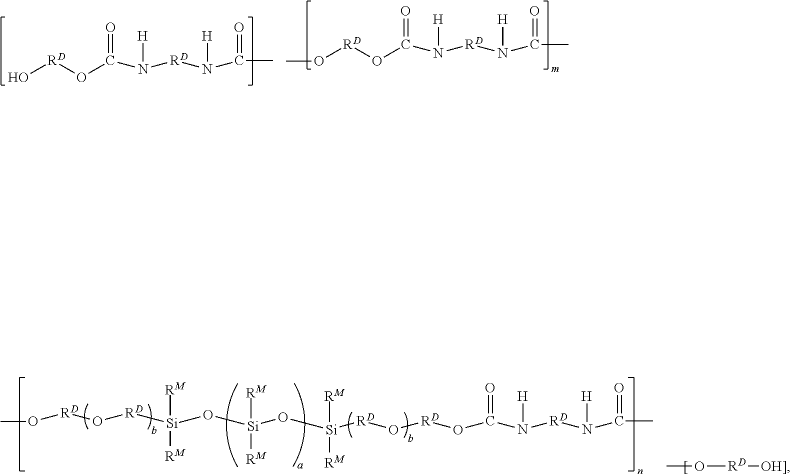

where R^{D} and R^{M} are as described above. Each subscript a is independently 0 to 1,000,000, each subscript m is independently greater than or equal to 0, each subscript b is independently greater than or equal to 0, and subscript n is greater than or equal to 1. Alternatively, each subscript b≥0. Alternatively, subscript b is 0 to 1,000,000. Alternatively, subscript b is 0 to 200,000. Alternatively, subscript b is 0 to 100,000. Alternatively, subscript b is 0 to 50,000. Alternatively, subscript b is 0 to 10,000. Alternatively, subscript b is 0 to 5,000. Alternatively, subscript b is 0 to 1,000. Alternatively, subscript b is 0 to 500. Alternatively, subscript b is 0 to 100. Alternatively, subscript b is 1 to 100. Alternatively, subscript b is 1 to 50. Alternatively, subscript b is 1 to 20. Alternatively, subscript b is 0 to 1. Alternatively, subscript b=0. Alternatively, subscript b=1. Alternatively, subscript b=2. Alternatively, subscript b=3. Alternatively, subscript b=4. Alternatively, subscript b=5.

Alternatively, copolymer (A) may have formula (II):

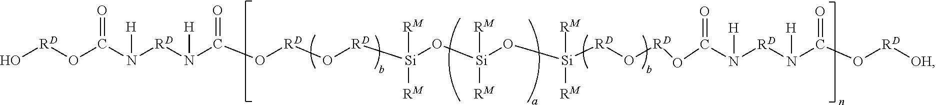

where R^{D} and R^{M} are as described above, subscript a is independently 0 to 1,000,000, each subscript b is independently greater than or equal to 0, and subscript n is greater than or equal to 1. Alternatively, each subscript b≥0. Alternatively, subscript b is 0 to 1,000,000. Alternatively, subscript b is 0 to 200,000. Alternatively, subscript b is 0 to 100,000. Alternatively, subscript b is 0 to 50,000. Alternatively, subscript b is 0 to 10,000. Alternatively, subscript b is 0 to 5,000. Alternatively, subscript b is 0 to 1,000. Alternatively, subscript b is 0 to 500. Alternatively, subscript b is 0 to 100. Alternatively, subscript b is 1 to 100. Alternatively, subscript b is 1 to 50. Alternatively, subscript b is 1 to 20. Alternatively, subscript b is 0 to 1. Alternatively, subscript b=0. Alternatively, subscript b=1. Alternatively, subscript b=2. Alternatively, subscript b=3. Alternatively, subscript b=4. Alternatively, subscript b=5.

### Copolymer (B)

Copolymer (B) is a siloxane-urethane-urea copolymer comprising units of formulae:

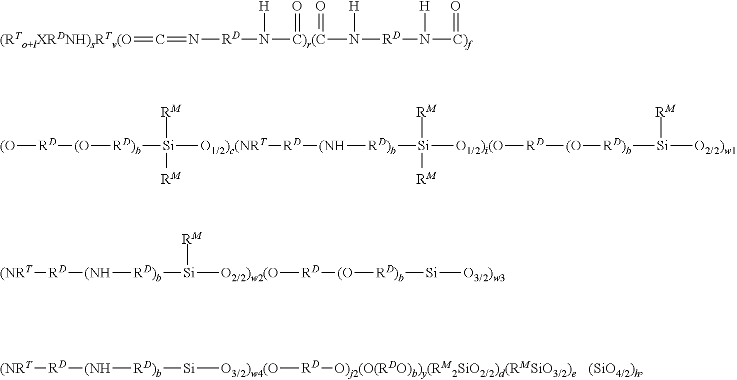

where R^{T}, R^{D}, R^{M}, and subscripts o, l, s, v, r, c, i, w1, w2, w3, w4, b, and y are as defined above for copolymer (A) and subscript j2 is >0. When copolymer (A) and copolymer (B) are both present in the copolymer composition, and when j1 is >0, then j2/j1 is ≥1.1. Alternatively subscript j2 is 1 to 500,000. Alternatively, subscript j2 is 1 to 200,000, alternatively 20 to 100,000, alternatively 50 to 50,000, alternatively 100 to 10,000, alternatively 1,000 to 5,000, alternatively 100 to 1,000, alternatively 10 to 500, and alternatively 15 to 200.

Alternatively, copolymer (B) may have unit formula (III):

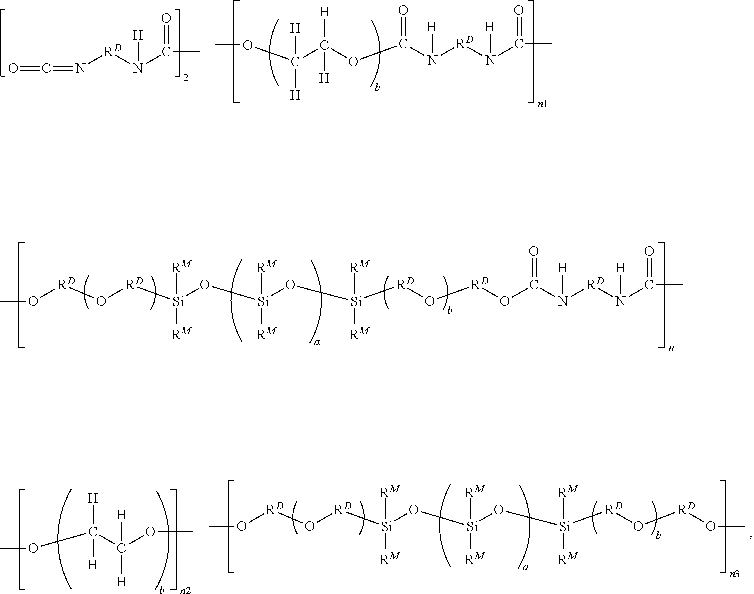

where R^{D}, R^{M}, subscripts a, b, and n are as described above, and subscript n1 is greater than or equal to 0, alternatively from 0 to 200,000, alternatively 0 to 20,000, alternatively 0 to 10,000, alternatively 0 to 5,000, alternatively 0 to 1,000, alternatively 0 to 100, alternatively 1 to 50. Subscripts n2 and n3 are each 0 or 1, and a quantity (n2+n3)=1.

### (C) Organic Polyol

Starting material (C) is an organic polyol. Suitable organic polyols are organic polymers containing two or more hydroxyl groups. The organic polyol for starting material (C) may be a polyether polyol, a polyester polyol, a polyacrylate polyol, a polycaprolactone polyol, a polyurethane polyol, a polycarbonate polyol, polybutadiene diol, other polymer polyols, or two or more of these organic polyols. Copolymer polyols of two or more types of polymers can also be used. Polyols with other modifications on the polymer structures, such as fluorination, can also be used. Suitable organic polyols alternatively may be an organic polymer diol. Such organic polymer diols include polyalkylene oxide diols e.g., polyethylene oxide diols, polypropylene oxide diols, and polybutylene oxide diols; or polycarbonate diols. Suitable organic polyols alternatively may be small molecule organic diols. Such small molecule organic diols include glycerol. The organic polyol may be added to tune the surface energy and/or hydrophilicity/mechanical properties of the copolymer composition. The amount added may be 0 to 95%, alternatively 0 to 75%, alternatively 0 to 50%, and alternatively 1 to 25%.

### (D) Reaction Product of an Organic Polyisocyanate and an Organic Polyol

Starting material (D) can be prepared by reacting starting material (C), the organic polyol described above, with an isocyanate compound, which has an average of one or more isocyanate groups per molecule. Alternatively, the organic isocyanate compound may have an average of two or more isocyanate groups per molecule. The organic isocyanate compound may have formula: R—(N═C═O)ₚ, where R is a hydrocarbon group or a halogenated hydrocarbon group and subscript p is an integer representing the number of isocyanate groups per molecule, and p is greater than or equal to 1. Alternatively subscript p is 2, 3, or 4; alternatively subscript p is 2 or 3; and alternatively, subscript p is 2. R is a divalent hydrocarbon group when subscript p is 2. R is a trivalent hydrocarbon group when subscript p is 3. R is a tetravalent hydrocarbon group when subscript p is 4.

The organic isocyanate compound is exemplified by monomeric isocyanates and polymeric isocyanates. Monomeric isocyanates include aromatic diisocyanates such as, meta-tetramethyl xylene diisocyanate (TMXDI), toluene diisocyanate (TDI), phenylene diisocyanate, xylene diisocyanate, 1,5-naphthalene diisocyanate, chlorophenylene 2,4-diisocyanate, bitoluene diisocyanate, dianisidine diisocyanate, toluidine diisocyanate and alkylated benzene diisocyanates; aliphatic and cycloaliphatic isocyanates such as hexamethylene diisocyanate (HDI), hydrogenated methylene diphenyl diisocyanate (HMDI), 1-isocyanato-3-isocyanatomethyl-3,5,5-trimethyl-cyclohexane (isophorone diisocyanate, IPDI), and nonanetriisocyanate (TTI), methylene-interrupted aromatic diisocyanates such as methylene-diphenyl-diisocyanate, especially the 4,4′-isomer (MDI) including alkylated analogs such as 3,3′-dimethyl-4,4′-diphenyl-methane diisocyanate; hydrogenated materials such as cyclohexylene diisocyanate, 4,4′-methylenedicyclohexyl diisocyanate; mixed aralkyl diisocyanates such as the tetramethylxylyl diisocyanates, 1,4-bis(1-isocyanato-1,1′-dimethylmethyl) benzene OCNC(CH₃)₂C₆H₄C(CH₃)₂NCO, and polymethylene isocyanates such as 1,4-tetramethylene diisocyanate, 1,5-pentamethylene diisocyanate, 1,6-hexamethylene diisocyanate (HDI), 1,7-heptamethylene diisocyanate, 2,2,4- and 2,4,4-trimethylhexamethylene diisocyanate, 1,10-decamethylene diisocyanate, and 2-methyl-1,5-pentamethylene diisocyanate; vinylisocyanate; and combinations thereof.

Polymeric organic isocyanates include dimerized isocyanates uretdiones or uretidinediones and carbodiimide, trimerized isocyanates isocyanurates, iminooxadiazine dione, uretonimine, and linear polymer α-Nylon; and derivatized isocyanates by reacting difuntional or multifunctional isocyanates with various compounds to form allophanate, or biuret compounds, or isocyanate functional urethane or other prepolymers. Some of the polyisocyanates are difunctional, i.e., having 2 isocyanate groups per molecule. Some have more than two isocyanate groups. An example is polymeric diphenylmethane diisocyanate, which is a mixture of molecules with two-, three-, and four- or more isocyanate groups, which may have an average functionality greater than two, commonly 2.7. Isocyanate functional compounds with isocyanate functionality greater than two may act as crosslinking sites. Commercially available isocyanate functional organic compounds are illustrated by Tolonate XIDT 70SB, an isophorone diisocyanate trimer (70% solids, 12.3 wt % NCO) sold by Rhodia (Cranbury, N.J.) and Desmodur N-100 polyisocyanate (available from Mobay Corp.).

The organic isocyanate compound can alternatively be a blocked isocyanate. The isocyanate group can be blocked by common blocking agents such as phenol, nonyl phenol, butanone oxime, caprolactam, and others. These blocked isocyanates can be release at a certain temperature to react with chain extenders and polyorganosiloxanes to construct an organic-siloxane copolymer. The blocking agent can react off/be released by heating to a certain temperature.

The reaction product (D) can be a low molecular weight compound, or a pre-polymer with low to medium molecular weight, or a high molecular weight polymer, depending on the isocyanate/OH reactive group molar ratio and the extent to which the reaction is carried out. The organic polyols can have a relatively large molecular weight and low glass transition temperature (Tg) so as to form a part of “soft segments” in the reaction product, or a low molecular weight so as to form the “hard segments” in the reaction product. Depending on the molar ratios of the polyols to isocyanates and the reaction, the reaction product (D) may have residual hydroxyl groups, or isocyanate groups, or both isocyanate group and hydroxyl groups, or no residual reactive groups.

The method of making reaction product (D) from the starting materials (i.e., organic polyol and polyisocyanate) are known, and any conventional method to make a polyurethane polymer can be employed. Such methods can be found in U.S. Pat. Nos. 3,384,623; 5,200,491; and 5,621,024. Method for making copolymer (A)

The method to make copolymer (A) is similar to the method to make the reaction product (D). The methods described in the references cited above may be used but varying the starting materials to those described herein.

The copolymer described above as starting material (A) may be prepared by a method comprising

1) reacting starting materials comprising:
a) an isocyanate compound,
b) a polyorganosiloxane, and
c) a chain extender.

In one embodiment, all starting materials may be combined and reacted concurrently. Alternatively, starting materials comprising a) the isocyanate compound and b) the polyorganosiloxane may be reacted to form a prepolymer, and thereafter the prepolymer may be reacted with a starting material comprising c) the chain extender, and optionally with an additional amount of a) the isocyanate compound to form the copolymer. Alternatively, starting materials comprising a) the isocyanate compound and c) the chain extender may be reacted to form an intermediate, and thereafter the intermediate may be reacted with a starting material comprising b) the polyorganosiloxane and optionally an additional amount of a) the isocyanate compound to form the copolymer.

In all these embodiments, wherever a polyorganosiloxane is reacted, a mixture of polyorganosiloxane and an organic polyol can be used in place of the polyorganosiloxane. Alternatively, the method may comprise: i) reacting a) the isocyanate compound with b) the polyorganosiloxane and d) an organic polyol to form a prepolymer, and thereafter ii) reacting the prepolymer with c) the chain extender, and optionally an additional amount of a) the isocyanate compound.

In each embodiment of the method described above, starting material b) the polyorganosiloxane may be b1) a carbinol-functional polyorganosiloxane, b2) an amine-functional polyorganosiloxane, or a mixture of both b1) and b2).

### Starting Material a) Isocyanate Compound

In the method described above, a) the isocyanate compound has an average of one or more isocyanate groups per molecule. Alternatively, the isocyanate compound may have an average of two or more isocyanate groups per molecule. The isocyanate compound may have formula: R—(N═C═O)ₚ, where R is a polyvalent hydrocarbon group or a polyvalent halogenated hydrocarbon group and subscript p is an integer representing the number of isocyanate groups per molecule. Subscript p is greater than or equal to 1. Alternatively, subscript p is 2, 3, or 4; alternatively subscript p is 2 or 3; and alternatively, subscript p is 2. R is a divalent hydrocarbon group when subscript p is 2. R is a trivalent hydrocarbon group when subscript p is 3. R is a tetravalent hydrocarbon group when subscript p is 4.

The isocyanate compound is exemplified by monomeric isocyanates and polymeric isocyanates. Monomeric isocyanates include aromatic diisocyanates such as meta-tetramethyl xylene diisocyanate (TMXDI), toluene diisocyanate (TDI), phenylene diisocyanate, xylene diisocyanate, 1,5-naphthalene diisocyanate, chlorophenylene 2,4-diisocyanate, bitoluene diisocyanate, dianisidine diisocyanate, toluidine diisocyanate and alkylated benzene diisocyanates; aliphatic and cycloaliphatic isocyanates such as hexamethylene diisocyanate (HDI), hydrogenated methylene diphenyl diisocyanate (HMDI), 1-isocyanato-3-isocyanatomethyl-3,5,5-trimethyl-cyclohexane (isophorone diisocyanate, IPDI), and nonanetriisocyanate (TTI), methylene-interrupted aromatic diisocyanates such as methylene-diphenyl-diisocyanate, especially the 4,4′-isomer (MDI) including alkylated analogs such as 3,3′-dimethyl-4,4′-diphenyl-methane diisocyanate; hydrogenated materials such as cyclohexylene diisocyanate, 4,4′-methylenedicyclohexyl diisocyanate; mixed aralkyl diisocyanates such as the tetramethylxylyl diisocyanates, 1,4-bis(1-isocyanato-1,1′-dimethylmethyl) benzene OCNC(CH₃)₂C₆H₄C(CH₃)₂NCO, and polymethylene isocyanates such as 1,4-tetramethylene diisocyanate, 1,5-pentamethylene diisocyanate, 1,6-hexamethylene diisocyanate (HDI), 1,7-heptamethylene diisocyanate, 2,2,4- and 2,4,4-trimethylhexamethylene diisocyanate, 1,10-decamethylene diisocyanate, and 2-methyl-1,5-pentamethylene diisocyanate, vinylisocyanate; and combinations thereof.

Polymeric isocyanates include dimerized isocyanates, uretdiones or uretidinediones, and carbodiimide, trimerized isocyanates, isocyanurates, iminooxadiazine dione, uretonimine, and linear polymer α-Nylon; and derivatized isocyanates by reacting difunctional or multifunctional isocyanates with various compounds to form allophanate, or biuret compounds, or isocyanate functional urethane or other prepolymers. Some of the polyisocyanates are difunctional, i.e., having 2 isocyanate groups per molecule. Some have more than two isocyanate groups. An example is polymeric diphenylmethane diisocyanate, which is a mixture of molecules with two-, three-, and four- or more isocyanate groups, which may have an average functionality greater than two, commonly 2.7. Isocyanate functional compounds with isocyanate functionality greater than two may act as crosslinking sites. Commercially available isocyanate functional organic compounds are illustrated by Tolonate XIDT 70SB, an isophorone diisocyanate trimer (70% solids, 12.3 wt % NCO) sold by Rhodia (Cranbury, N.J.) and Desmodur N-100 polyisocyanate (available from Mobay Corp.). Alternatively, a) the isocyanate compound may comprise a blocked isocyanate. The isocyanate group can be blocked by common blocking agents such as phenol, nonyl phenol, butanone oxime, caprolactam, and others. These blocked isocyanates can be released by any conventional means such as heating at a temperature above room temperature to react with chain extenders and polyorganosiloxanes to construct the polyurethane-polyorganosiloxane copolymer.

### Starting Material b1) Carbinol-Functional Polyorganosiloxane

In the method described above, b1) the carbinol-functional polyorganosiloxane comprises units of formulae:

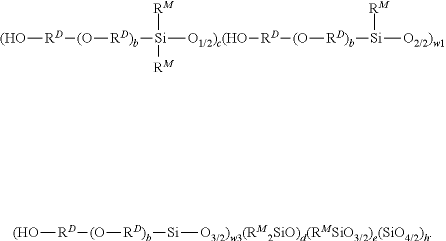

In this unit formula, each R^{M}, R^{D}, subscript b, subscript c, subscript w1, subscript w3, subscript d, subscript e, and subscript h are as described above. Examples of carbinol-functional polyorganosiloxanes are disclosed in WO2008/088491, U.S. Pat. Nos. 6,528,121, and 7,452,956. The carbinol groups can be terminal or pendent. Alternatively, the carbinol groups may be terminal. Alternatively, b1) the carbinol-functional polyorganosiloxane may comprise an α,ω-difunctional polydiorganosiloxane of formula (II): R^{C}R^{M}₂Si—R^{DX}—(R^{M}₂SiO)ᵣ—(R^{M}₂)SiR^{DX}—SiR^{M}₂R^{C}, where, each R^{C} is independently a carbinol functional group of formula HO—R^{D}—(OR^{D})_{b}— where subscript b, R^{M} and R^{D} are as described above, each R^{DX} is independently selected from 0 or a divalent hydrocarbon group described above as R^{D}, and subscript r represents the degree of polymerization of the carbinol-functional polyorganosiloxane of formula (II). Subscript r>0. Alternatively, subscript r may be 1 to 1,000,000, alternatively 50 to 1,000, and alternatively 200 to 700. Alternatively, subscript r is 0 to 200,000, alternatively 0 to 200,000, alternatively 0 to 100,000, alternatively 0 to 50,000, alternatively 0 to 10,000, alternatively 0 to 5,000, alternatively 0 to 1,000, alternatively 1 to 1,000, alternatively 1 to 500, alternatively 1 to 200, and alternatively 5 to 150. Alternatively, each R^{DX} is 0.

### Starting Material b2) Amine-Functional Polyorganosiloxane

The amine functional polyorganosiloxane comprises units of formulae:

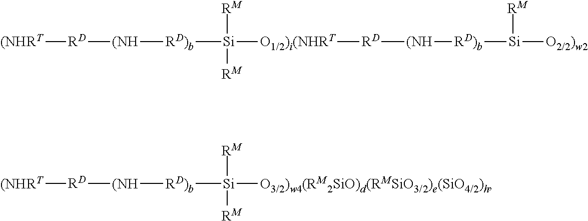

where R^{M} and R^{D}, and subscripts b, d, e, h, and i are as described above. The amine groups can be terminal or pendent. Alternatively, the amine groups can be terminal.

An exemplary amine terminated polyorganosiloxane comprises a terminal unit of formula

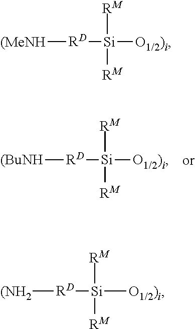

where Me represents a methyl group and Bu represents a butyl group; and further comprises units comprising one or more of (R^{M}₂SiO_{2/2})_{d}(R^{M}SiO_{3/2})ₑ(SiO_{4/2})ₕ, where R^{M}, R^{D}, and subscripts i, d, e, and h are as described above.

### Starting Material c) Chain Extender

The chain extender may be a dialcohol, of formula HO—R^{D}—OH, where R^{D} is as defined above. Suitable dialcohols include 1,3-butanediol; 1,4-butanediol; 1,6-hexanediol, 1,10-decanediol; 1,6-hexamethylenediol; 2,2-dimethyl-1,3-propanediol; 1,4-cyclohexanedimethylol; 1,1′-isopropylidine-bis-(p-phenylene-oxy)-di-2-ethanol; poly(tetrmethylene ether) glycol; and ethylene glycol. Alternatively, the chain extender may be a diamine containing 2 to 20 carbon atoms e.g., 1,2-diaminoethane; 1,4-diaminobutane; 1,2-propanediamine; hexamethylenediamine; diethylene diamine; 5-amino-1-(aminomethyl)-1,3,3-trimethylcyclohexane; 4,4′-methylene bis(cyclohexylamine); and ethanol amine. Alternatively, the chain extender may be a dithiol, a dicarboxylic acid, or a diepoxide. Suitable chain extenders are disclosed, for example, in U.S. Pat. Nos. 4,840,796 and 5,756,572.

### Starting Material d) Solvent

A solvent may be added during the method to prepare a copolymer described herein. Any organic compound that will dissolve the copolymer and that is relatively unreactive towards isocyanate, and amine and/or carbinol compounds is suitable as a solvent. Examples include aliphatic hydrocarbons, aromatic hydrocarbons, esters, ethers, ketones, and amides. Exemplary solvents include methyl ethyl ketone, ethyl acetate, butyl acetate, or tetrahydrofuran.

The amount of solvent to be used depends on the properties of the copolymer including structure, molecular weight, and the particular method of copolymer preparation, and can be 0 to 99%. Generally for higher molecular weight copolymers especially when a high torque mixing mechanism will not be used, solvent may be added to reduce the viscosity and make the system easier to handle during performance of the method to make the copolymer. If the molecular weight is relatively low and/or high torque mixing equipment such as a twin screw extruder is used, no solvent needs to be used. When solvent is used, the amount may be 0 to 99%, alternatively 0 to 80%, alternatively 1% to 60%, and alternatively 5% to 50%, based on the combined weights of all starting materials used.

The amounts of starting materials a), b), c), and when present, d) and/or e), can vary widely, according to the polyorganosiloxane structure and molecular weight desired, to arrive at the copolymer described by the formula herein. The molar ratio of isocyanate groups of starting material a) to the active hydrogen of carbinol or amine groups on the polysiloxane selected for starting material b) can be 0.1 to 100, alternatively 0.1 to 50, alternatively 0.1 to 10, alternatively 0.1 to 2, alternatively 0.1 to 1.5, alternatively 0.1 to 1.25, alternatively 0.1 to 1.1, alternatively 0.1 to 1.05, alternatively 0.1 to 1.01, alternatively 0.1 to 1, alternatively 0.1 to 0.9, alternatively 0.1 to 0.5, alternatively 0.5 to 50, alternatively 0.5 to 10, alternatively 0.5 to 2, alternatively 0.5 to 1.5, alternatively 0.5 to 1.25, alternatively 0.5 to 1.1, alternatively 0.5 to 1.05, alternatively 0.5 to 1.01, alternatively 0.5 to 1, alternatively 0.5 to 0.9, and alternatively 0.4 to 0.7.

The molar ratio between the isocyanate groups to the active hydrogen on the hydroxyl or amine groups or other reactive groups on the chain extender can be 1.001 to 1,000,000, alternatively 1.001 to 500,000, alternatively 1.001 to 200,000, alternatively 1.001 to 100,000, alternatively 1.001 to 50,000, alternatively 1.001 to 10,000, alternatively 1.001 to 5,000, alternatively 1.001 to 1,000, alternatively 1.001 to 500, alternatively 1.001 to 100, alternatively 1.001 to 50, alternatively 1.001 to 20, alternatively 1.001 to 10, alternatively 1.001 to 5, alternatively 1.001 to 4, alternatively 1.001 to 3, alternatively 1.001 to 2, alternatively 1.001 to 1.5, alternatively 1.001 to 1.3, alternatively 1.001 to 1.2, alternatively 1.01 to 20, alternatively 1.01 to 10, alternatively 1.01 to 5, alternatively 1.01 to 4, alternatively 1.01 to 3, alternatively 1.01 to 2, alternatively 1.01 to 1.5, alternatively 1.01 to 1.3, and alternatively 1.01 to 1.2.

### Starting Material e) Catalyst

Reacting the starting materials comprising a), b), and c) described above may be catalyzed by starting material e) a catalyst. Suitable catalysts include tertiary amines and metal salts, for example, the salts of tin. Tin compounds are useful as catalysts herein include those where the oxidation state of the tin is either +4 or +2, i.e., tin (IV) compounds or tin (II) compounds. Examples of tin (IV) compounds include stannic salts such as dibutyl tin dilaurate, dimethyl tin dilaurate, di-(n-butyl)tin bis-ketonate, dibutyl tin diacetate, dibutyl tin maleate, dibutyl tin diacetylacetonate, dibutyl tin dimethoxide, carbomethoxyphenyl tin tris-uberate, dibutyl tin dioctanoate, dibutyl tin diformate, isobutyl tin triceroate, dimethyl tin dibutyrate, dimethyl tin di-neodecanoate, dibutyl tin di-neodecanoate, triethyl tin tartrate, dibutyl tin dibenzoate, butyltintri-2-ethylhexanoate, dioctyl tin diacetate, tin octylate, tin oleate, tin butyrate, tin naphthenate, dimethyl tin dichloride, a combination thereof, and/or a partial hydrolysis product thereof. Tin (IV) compounds are known in the art and are commercially available, such as Metatin® 740 and Fascat® 4202 from Acima Specialty Chemicals of Switzerland, Europe, which is a business unit of The Dow Chemical Company. Examples of tin (II) compounds include tin (II) salts of organic carboxylic acids such as tin (II) diacetate, tin (II) dioctanoate, tin (II) diethylhexanoate, tin (II) dilaurate, stannous salts of carboxylic acids such as stannous octoate, stannous oleate, stannous acetate, stannous laurate, stannous stearate, stannous naphthanate, stannous hexanoate, stannous succinate, stannous caprylate, and a combination thereof. Other metal salts are also suitable catalysts for this reaction. Examples include zinc salts such as zinc acetate and zinc naphthenate. Salts of lead, bismuth, cobalt, iron, antimony, or sodium, such as lead octoate, bismuth nitrate, and sodium acetate can also catalyze this reaction. In certain occasions organomercuric compounds can also be used. Optionally co-catalysts can also be used along with a catalyst described above. Alternatively, a combination of two or more catalysts can be used, e.g., to provide either faster reaction than achievable with a single catalyst, or a better balanced reaction initiation time and finish time.

### Starting Material f) Organic Polyol

An organic polyol may optionally be combined with b) the polyorganosiloxane to make the copolymer (A) and/or copolymer (B) described above. Suitable organic polyols are organic polymers containing two or more hydroxyl groups. The organic polyol for starting material f) may be a polyether polyol, a polyester polyol, a polyacrylate polyol, a polycaprolactone polyol, a polyurethane polyol, a polycarbonate polyol, polybutadiene diol, other polymer polyols, or two or more of these organic polyols. Copolymer polyols of two or more types of polyolscan also be used. Polymeric polyols with other modifications on the polymer structures, such as fluorination, can also be used. Suitable organic polyols alternatively may be organic diols. Suitable organic diols include polyalkylene oxide diols e.g., polyethylene oxide diols, polypropylene oxide diols, and polybutylene oxide diols; or polycarbonate diols. Other organic diols include glycerol. The organic polyol may be added to tune the surface energy and/or hydrophilicity/mechanical properties of the copolymer being prepared. The amount added may be 0 to 95%, alternatively 0 to 75%, alternatively 0 to 50%, and alternatively 1 to 25% based on combined weights of all starting materials used to make the copolymer.

### Starting Material q) Optional Enblocker.

The copolymer (A) and/or (B) described above may optionally be reacted with an enblocker to convert any residual isocyanate groups, hydroxyl groups, or amine groups to another type of reactive or non-reactive group. Suitable endblockers include but are not limited to alcohols such ethanol, propanol, butanol, carboxylic acids such as acetic acids, and alcohols and carboxylic acids containing aliphatic unsaturation. Thio-alcohols, hydroxylamines, glycol, aminoacids, and amino sugars are also suitable as endblockers.

### Method for Making Copolymer (B).

The same method for making copolymer (A) can be used to make copolymer (B), except that the ratios of the starting materials will change to arrive at the desired composition for copolymer (B). One skilled in the art would recognize that copolymer (A) and copolymer (B) are chosen to be distinct from one another. Copolymer (A) and copolymer (B) differ from one another in at least one property such as structure, selection of copolymer units, sequence of the copolymer units, and molecular weight.

### Method Conditions for Making the Copolymers

The method described above may be performed with or without heating. The temperature for the reaction depends on the selection of starting materials a), b), and c) and whether any of d), e), f), and/or g) is present, however, the temperature may range from −20° C. to 150° C.; alternatively 0° C. to 100° C., and alternatively 20° C. to 60° C. at pressure of 1 atmosphere. Pressure under which the method is performed is not critical.

The method described above may be performed in batch, semi-batch, semi-continuous, or continuous mode in any convenient equipment. When preparing higher molecular weight copolymers (e.g., when higher molecular weight starting materials are used), the method may be performed in an extruder, such as a twin screw extruder. The copolymer described above may be prepared using the equipment and method as described in U.S. Pat. No. 5,756,572, except using the starting materials described above.

### Skin Contact Adhesive

The skin contact adhesive may be prepared by a method comprising hardening the copolymer composition described above. Hardening may be performed by any convenient means, such as cooling the composition to room temperature of 15° C. to 40° C. and/or removing solvent from the composition. The skin contact adhesive prepared by hardening the copolymer composition is useful in applications such as adhesives for medical tapes, adhesives for wound dressings, adhesives for prosthetics, ostomy appliance adhesives, adhesives for medical monitoring appliances, adhesives for cosmetic patches, adhesives for scar therapy treatments, and transdermal drug delivery systems.

The skin contact adhesive composition and skin contact adhesive described above comprises (I) the copolymer composition described above. The skin contact adhesive composition may further comprise (II) an excipient. The skin contact adhesive composition and skin contact adhesive described above may optionally further comprise (III) an active ingredient.

### (II) Excipient

The excipient may be any ingredient that is distinct from ingredients (I) and (III) and that is added to the composition to provide one or more benefits during and/or after making the composition and/or to provide one or more benefits to the skin contact adhesive. For example, the excipient may be (II-1) a stabilizer, (II-2) a binder, (II-3) a filler, (II-4) a solubilizer, (II-5) a skin penetration enhancer, (II-6) an adhesion promoter, (II-7) agent to improve moisture permeability, or a combination of two or more of (II-1), (II-2), (II-3), (II-4), (II-5), (II-6), and (II-7).

### (II-1) Stabilizer

The composition may optionally further comprise (II-1) a stabilizer. The stabilizer may comprise an antioxidant, such as vitamin A, vitamin E, vitamin C, retinyl palmitate, selenium, benzenepropanoic acid, 3,5-bis(1,1dimethyl-ethyl)-4-hycroxy-C7-C9 branched alkyl esters (Irganox® 1135 from BASF), pentaerythritoltetrakis[3-(3,5-di-tert-butyl-4-hydroxyphenyl) propionate] (Irganox® 1010 from BASF), octadecyl-3-(3,5-di-tert-butyl-4-hydroxyphenyl)propionate (Irganox® 1076 from BASF), 1,3,5-Trimethyl-2,4,6-tris(3,5-di-tert-butyl-4-hydroxybenzyl)benzene (Irganox® 1330 also from BASF), 2-methyl-4,6-bis[(octylthio)methyl]phenol (Irganox® 1520 from BASF) 2,6-di-tert-butyl-methylphenol (BHT), 2,2′-methylene-bis-(6-tert-butyl-4-methylphenol) (Vulkanox BKF from LanXess), or mixtures thereof. Alternatively, the stabilizer may comprise an amino acid such as cysteine, methionine, or combinations thereof. Alternatively, the stabilizer may comprise a paraben, such as methyl paraben, propyl paraben, or combinations thereof. The amount of stabilizer depends on various factors including whether the composition will be heated and whether ingredient (III) will be added, however, the stabilizer may be present in an amount from 0 to 2%, alternatively 0 to 1%, alternatively 0.1% to 1%, alternatively 0.2% to 0.7%, and alternatively 0.2% to 0.6% based on the weight of the composition.

### (II-2) Binder

Ingredient (II-2), a binder, may optionally be added to the composition. Suitable binders include saccharides and their derivatives (e.g., disaccharides such as sucrose and lactose, polysaccharides such as starches or cellulose, or sugar alcohols such as xylitol, sorbitol or malitol. Other suitable binders include proteins such as gelatin. The amount of binder depends on various factors including the type of laminate article and the selection of other ingredients in the composition, however, the amount of binder may be 0 to 50% based on the weight of the composition.

### (II-3) Filler

Ingredient (II-3), a filler, may optionally be added to the composition. Suitable fillers for ingredient (II-3) include but are not limited to silica to help prevent cold flow of the composition off the support. The filler selected is of a type and is present in an amount so as not to detrimentally impact adhesion of the skin contact adhesive. The amount of filler may be 0 to 2%, alternatively 0 to 1%, based on the weight of the composition.

### (II-4) Solubilizer

Ingredient (II-4), a solubilizer, may optionally be added to the composition. Suitable solubilizers include dimethylsulfoxide, povidone (PVP) and natural oils such as mineral oil, sunflower oil, and peanut oil. Esters, glycols, polyether, may help solubilize (III) the active ingredient (i.e., keep ingredient (III) in a noncrystalline state in the composition, and the skin contact adhesive prepared therefrom, to facilitate permeation of the active ingredient to the skin (and/or into the skin). The solubilizer may be present at 0 to 50%, alternatively 0 to 40%, alternatively 0 to 25%, alternatively greater than 0 to 20%, and alternatively 20% to 25%, based on the weight of the composition. Alternatively, the solubilizer suitable for ingredient (II-4) may be the solvent, described above for making the copolymer.

### (II-5) Skin Penetration Enhancer

Ingredient (II-5), a skin penetration enhancer, may optionally be added to the composition. Suitable skin penetration enhancers include glycols such as propylene glycol and polyethylene glycol; organic acids such as oleic acid; fatty alcohols such as oleyl alcohol; and amines. The amount of ingredient (II-5) depends on various factors including where the skin contact adhesive prepared from the composition will be applied, the length of time the skin contact adhesive will be applied, and the purpose (e.g., wound dressing or transdermal drug delivery), however the amount may range from 0 to less than 20%, alternatively 1% to 2% based on the weight of the composition.

### (II-6) Adhesion Promoter

Materials known in the art as skin contact adhesives may be mixed with the composition described herein to adjust adhesive properties, such as release force required to remove the skin contact adhesive and amount of residue remaining on skin. These materials may be used herein as adhesion promoters. Exemplary adhesion promoters include hydrocolloids. The amount of adhesion promoter depends on the type of adhesion promoter selected and the amount of adhesion desired, however the amount of adhesion promoter may be 0 to less than 20%, alternatively 1% to 2%, based on the weight of the composition.

### (II-7) Agent to Improve Moisture Permeability

Ingredient (II-7) is an agent to improve moisture permeability, which may optionally be added to the composition. Suitable agents for ingredient (II-7) included but are not limited to hydrocolloids, gelatins, polymers such as CMC carboxymethylcellulose, and polyethylene oxide. The amount of ingredient (II-7) depends on various factors including the selection of the other ingredients in the composition and the end use for the skin contact adhesive prepared therefrom. However, the amount of ingredient (II-7) may be 0.1% to 50%, alternatively 0.1% to 25%, alternatively 0.1% to 10%, alternatively 1% to 10%, based on the weight of the composition. One skilled in the art would recognize that certain agents that improve moisture permeability may also act as mucoadhesives that make the dressing adhere better as moisture content increases.

When selecting ingredients for the composition described above, there may be overlap between types of ingredients because certain ingredients described herein may have more than one function. For example, certain hydrocolloids may be useful as agents to improve moisture permeability (II-7) and as adhesion promoters (II-6). Gelatin may be useful as an agent to improve moisture permeability (II-7) and as a binder (II-2). Certain nutrients such as vitamin A and vitamin E may be useful as a stabilizer (II-1) and as an active ingredient (III). When adding ingredients to the composition, the ingredients are distinct from one another.

### (III) Active Ingredient

The composition optionally further comprise (III) an active ingredient. Ingredient (III) may be added, for example, when the composition will be used to prepare a skin contact adhesive in a scar treatment application, a cosmetic patch application, a transdermal drug delivery application, and/or in an application for delivery of the active ingredient to the skin. The specific active ingredients used are not critical to this invention and as used herein the term “active ingredient” is to be construed in its broadest sense as a material intended to produce some beneficial effect on the organism to which it is applied.

Exemplary active ingredients suitable for ingredient (III) include, without limitation, drugs that act upon the central nervous system, drugs affecting renal function, drugs affecting cardiovascular function, drugs affecting gastrointestinal function, drugs for treatment of helminthiasis, antimicrobial agents such as silver, silver compounds, and/or chlorhexidine, nutrients, hormones, steroids, and drugs for treatment of dermatoses; see for example, those disclosed in U.S. Patent Application Publication US2007/0172518 paragraph [0014] and those listed in PCT Publication WO2007/092350 at pp. 21-28.

Other suitable active ingredients for ingredient (III) include non-steroidal anti-inflammatory drugs such as salicylates e.g., acetylsalicylic acid; propionic acid derivatives e.g., (RS)-2-(4-(2-Methylpropyl)phenyl)propanoic acid (ibuprofen); acetic acid derivatives e.g., 2-{1-[(4-Chlorophenyl)carbonyl]-5-methoxy-2-methyl-1H-indol-3-yl}acetic acid (indomethacin), enolic acid derivatives; anthranilic acid derivatives, COX-2 inhibitors e.g., N-(4-hydroxyphenyl)ethanamide N-(4-hydroxyphenyl)acetamide (acetaminophen), and sulfonanilides. Other suitable active ingredients for ingredient (III) include local anesthetics such those containing an ester group e.g., ethyl 4-aminobenzoate (benzocaine); those containing an amide group e.g., 2-(diethylamino)-N-(2,6-dimethylphenyl)acetamide (lidocaine); and naturally derived local anesthetics e.g., (1R,2S,5R)-2-Isopropyl-5-methylcyclohexanol (menthol).

One skilled in the art would recognize that in the laminate articles described below, (III) the active ingredient may be included in the skin contact adhesive prepared by including ingredient (III) in the composition described herein (i.e., before hardening said composition to form the skin contact adhesive). Alternatively, (III) the active ingredient may be included in a separate reservoir within the laminate article, and not mixed into the skin contact adhesive prepared from the skin contact adhesive composition.

The amount of (III) the active ingredient used in the skin contact adhesive composition depends on various factors including the type of active ingredient selected for ingredient (III), the and type of laminate article in which the active ingredient will be incorporated, and the selection of any other ingredients in the composition. However, the amount of ingredient (III) may be 0 to 45%, alternatively greater than 0 to 25%, alternatively greater than 0 to 15%, alternatively greater than 0 to 10%, alternatively greater than 0.1% to 10%, alternatively greater than 1% to 10%, based on the weight of the skin contact adhesive composition.

### Laminate Article

A laminate article comprises:

i) a support having a skin facing surface and an opposed surface, which is intended to be facing away from skin,
ii) a skin contact adhesive on at least a portion of the skin facing surface, where the skin contact adhesive has a skin contact surface opposite the skin facing surface of the support.

The support is a material that can readily be applied to a part of the wearer's body. The support may be a plastic film, such as polyurethane, a polyolefin such as low density polyethylene (LDPE), medium density polyethylene (MDPE), high density polyethylene (HDPE), or polypropylene; a polyolefin/polyurethane composite; polyester; or ethylene vinyl acetate (EVA). Alternatively, the support may be paper, fabric (woven or nonwoven), silicone rubber, or foam. All, or a portion, of the support may optionally have a plurality of holes, e.g., be perforated or apertured, to provide for air permeability in the laminate article. Suitable supports are known, see for example PCT Publications WO2013/030580 and WO2014/116281 at pages 5-6.

The skin contact adhesive is on at least a portion of the skin facing surface of the support. For certain applications, such as transdermal drug delivery, the skin contact adhesive may cover all or most of the skin facing surface of the support to maximize the surface area through which the drug can be transferred. Alternatively, the skin contact adhesive may be on a portion of the skin facing surface of the support, for example, when the skin contact adhesive will be used to adhere an absorbent material to a wound. The amount (thickness) of the skin contact adhesive on the support will vary depending on various factors including the application (e.g., ostomy, wound care, and other applications where strong adhesion for longer time periods may have a thicker skin contact adhesive on the support, but adhesives for transdermal drug delivery or bandages or medical tapes may have a thinner skin contact adhesive on the support. Thickness may be uniform. Alternatively, thickness may be non-uniform on any given support, e.g., thicker toward the middle and thinner at or near the edge of the support). However, thickness of the skin contact adhesive may range from 0.0635 mm to 2.54 mm, alternatively 0.254 mm to 1 mm.

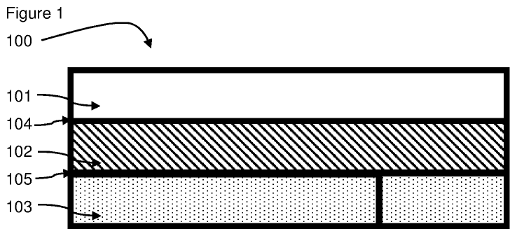

FIG. 1 is a partial cross section of a laminate article 100 according to this invention. The laminate article 100 comprises a support 101 having a layer of skin contact adhesive 102 on a skin facing surface 104 of the support 101. A release liner 103 covers the skin contacting surface 105 of the layer of skin contact adhesive 102. The support 101 may be a backing for a medical tape or adhesive bandage or other wound dressing, and is as described above.

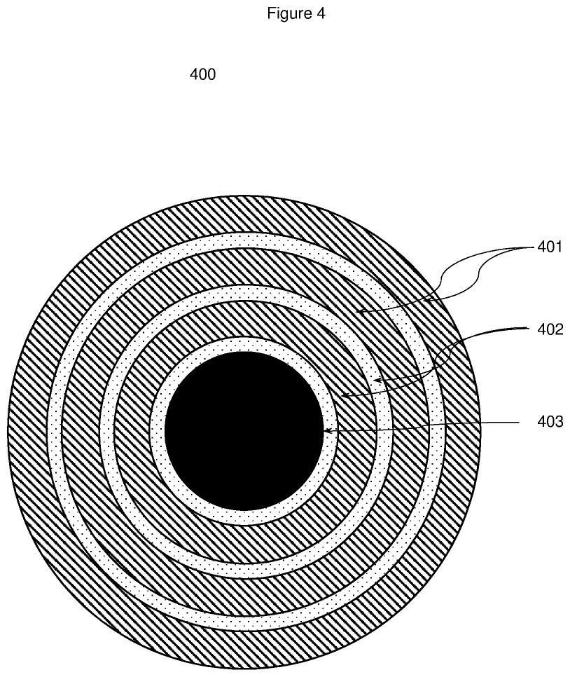

The layer of skin contact adhesive can be continuous or discontinuous. When discontinuous, the layer may be in various forms such as lines, line segments, dots, or flecks. The discontinuous forms may be in a uniform pattern across the surface of the support, or have different patterns at different regions of the support. An example is in FIG. 4, which shows a flange 400 for use in an ostomy appliance (not shown). The flange 400 has a support member 401 defining an aperture 403. The skin contact adhesive 402 described herein is formed in a discontinuous layer (shown as circular lines) on the support member 401.

The laminate article may further comprise one or more additional layers. For example, the laminate article may further comprise iii) a release liner covering the skin contact surface of the skin contact adhesive. The release liner is removable and may be used during shipping and storage of the laminate article before use. The skin contact adhesive can be exposed by removal of the release liner.

Suitable release liners include liners made of or coated with polyethylene, polypropylene, fluorocarbons, and fluorosilicone coated release papers and fluorosilicone coated plastic films. Suitable release liners are known and are described for example, in PCT Publication WO2007/092350. Without wishing to be bound by theory, it is thought that one benefit of the skin contact adhesive prepared by hardening the composition described herein is that release liners without fluorinated coatings (e.g., without fluorocarbons and without fluorosilicones) can be effectively used with the skin contact adhesive. Release liners with fluorinated coatings are typically more expensive than release liners without a fluorinated coating. Alternatively, release liners include liners made of or coated with polyethylene or polypropylene.

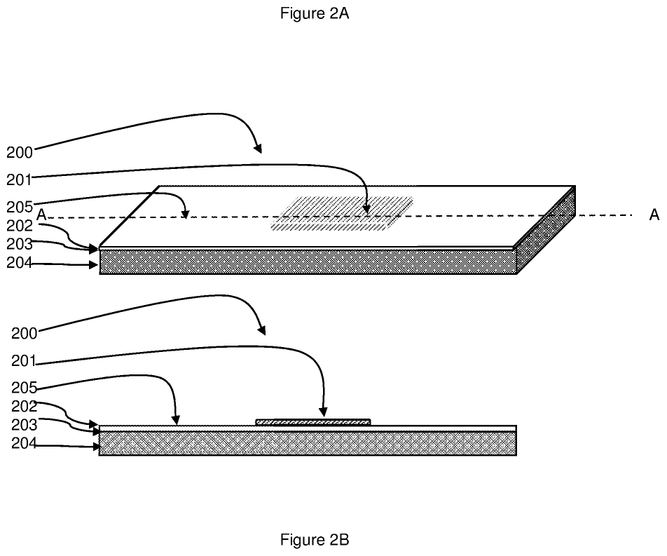

The laminate article may optionally further comprise iv) an absorbent layer. The absorbent layer may be mounted to the skin contact surface of the skin contact adhesive when the absorbent layer will contact the skin (e.g., a wound) directly, such as when the laminate article is an adhesive bandage such as that shown in FIG. 2 or in Canadian Patent Publication CA02585933. FIG. 2A shows a perspective view of an adhesive bandage 200 including a thin layer of the skin contact adhesive 202 described herein. FIG. 2B shows a cross sectional view of the adhesive bandage 200 taken along line A-A in FIG. 2A. The adhesive bandage 200 has a perforated plastic support 204 with the layer of the skin contact adhesive 202 on a skin facing surface 203 of the support 204. An absorbent layer 201 is on the skin contact surface 205 of the skin contact adhesive 202. Alternatively, the absorbent layer may be located between the skin contact adhesive and the support, for example, when the skin contact adhesive described herein is used in a wound dressing such as that shown in PCT Publication WO2007/092350.

The absorbent layer may be any suitable material such as a textile or polymer composition that is capable of absorbing fluid (e.g., exudate from a wound). The absorbent layer may be a commercially available product, see PCT Publication WO2007/092350 for examples of absorbent polymers, at pages 12 to 15. Examples include but are not limited to: thermoplastic polymers, block copolymers (other than ingredient (A)), polyolefins, hydrogels, and hydrocolloids.

The laminate article may further comprise v) a carrier. The carrier may be used to provide some rigidity to the laminate article and to enable the laminate article to be placed over a wound with minimal wrinkling and to avoid having the skin contact adhesive stick to itself during application of the laminate article to a wearer. The carrier may optionally be removed, e.g., after the laminate article is adhesively secured to the skin. The carrier may be mounted on the opposed surface of the support, intended to be facing away from the skin.

The carrier can be ethylene vinyl acetate (EVA), polyethylene film, polyester film, or paper coated with an EVA coating. One skilled in the art would recognize that the carrier may have the same materials of construction as the support, or different materials of construction. The carrier, as used herein, refers to a separate, discrete, piece of the laminate article.

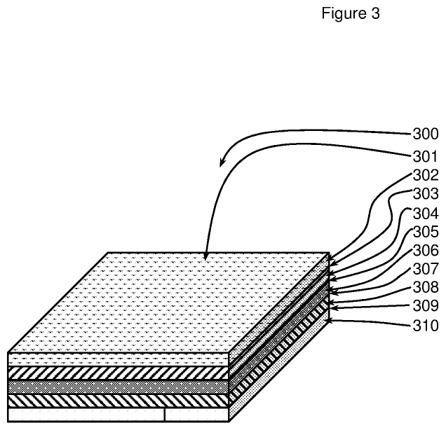

FIG. 3 is a partial cross section of an alternative laminate article 300 according to this invention. The laminate article 300 has a support 304 with a skin facing surface 305 and an opposed surface 303 intended to face away from the skin. The skin contact adhesive (described herein) 308 is mounted to the skin facing surface 305 of the support 304. The skin contact adhesive 308 forms a layer with a skin contact surface 309. A release liner 310 (with two parts that can be peeled away separately) covers the skin contact surface 309 of the skin contact adhesive 308. The laminate article further comprises an absorbent layer 306 between the skin facing surface 305 of the support 304 and the opposed surface 307 of the skin contact adhesive 308. The laminate article further comprises a carrier 302 having a skin facing surface and an opposed surface 301. The carrier 302 is removably affixed to the opposed surface 303 of the support 304.

### Method for Making a Laminate Article

A method for making the laminate article comprises:

I) forming a layer of the composition described above on at least a portion of a skin facing surface of a support, and
II) hardening the composition to form the skin contact adhesive.
The method may optionally further comprise: III) applying a release liner to a skin contact surface of the skin contact adhesive opposite the skin facing surface of the support. Step III) may be performed either before or after step II). The method may further comprise: IV) compressing the composition between the support and the release liner before hardening in step II).

The composition may be applied to support by any convenient means typically used for applying pressure sensitive adhesive to substrates. The composition may be applied by, e.g., melt coating, doctor blade drawdown techniques, solvent casting, knife coating or roll coating. The composition may be applied to the support or the release liner first. The composition may be applied to the support using the method described, for example, in U.S. Pat. No. 5,756,572 (substituting the composition described herein for the pressure sensitive adhesive described in the reference). Alternatively, the composition may be sandwiched between the support and release liners, and heat and/or pressure may be applied to form the laminate article and/or crosslink the composition to form the skin contact adhesive. Laminate articles may be prepared as described in WO2015/075448, except using the composition of this invention instead of the polyurethane gel adhesive formulation disclosed in the reference.

The method for making the laminate article may optionally further comprise: Ill) sterilizing the laminate article. The laminate article including the skin contact adhesive is capable of being sterilized. The laminate article may be sterilized using known sterilizing means such as irradiating (e.g., with electron beam or gamma radiation) and/or heating such as with dry heat or steam. Sterilizing in step III) may be performed as a separate step after step I) or step II), as described above. Alternatively, sterilizing may be performed concurrently with steps I) and/or step II). For example, heating and/or irradiating may be performed to crosslink the composition, remove solvent, and/or sterilize.

### Applications

The skin contact adhesive described herein is suitable for use in various applications. The skin contact adhesive prepared by crosslinking the composition is useful in applications such as adhesives for medical tapes, adhesives for wound dressings, adhesives for prosthetics, ostomy appliance adhesives, adhesives for medical monitoring appliances, adhesives for scar therapy treatments, and transdermal drug delivery systems.

For example, the laminate article described above may comprise the support and the skin contact adhesive described above, on all or a portion of a surface of the support. The skin contact adhesive may be formed in a layer which is continuous or discontinuous. In one embodiment, the laminate article described above may be useful as an adhesive element. The skin contact adhesive may be applied to a skin facing surface of a support, and the skin contact adhesive may be used to adhere the support to the skin of a wearer. For example, the skin contact adhesive described above may be used to adhere a prosthetic to a wearer with a limb difference, or the skin contact adhesive may be used to adhere an ostomy appliance to a patient with a stoma. An ostomy appliance typically comprises a pouch for collection of waste, which is attached to a flange defining an aperture. The flange has an adhesive on the skin facing surface, where the adhesive surrounds the opening for attachment to the skin of a patient with a stoma (as described above in FIG. 4).

The skin contact adhesive described herein may be used in would care and ostomy care applications for adhesion to the skin, instead of the pressure sensitive adhesive disclosed in U.S. Patent Application Publication US2005/0163978, or instead of the adhesive used in U.S. Patent Application Publication US2014/0323941.

The skin contact adhesive described herein is suitable for use in wound dressings. For example, the skin contact adhesive described herein may be used as the skin contacting barrier layer instead of the hydrocolloids in U.S. Pat. No. 5,998,694. The skin contact adhesive described herein may be used in the wound cover of PCT Publication WO2007/092350 and US Patent Application Publications US2009/0105670 and US2015/0313593.

Alternatively, the skin contact adhesive described herein may be used in a transdermal drug delivery system. In this embodiment, the composition described above comprises ingredient (III) the active ingredient and may further comprise (II) the excipient. Without wishing to be bound by theory, it is thought that this invention may provide the benefit that crosslinking the composition to form the skin contact adhesive does not detrimentally affect (III) the active ingredient. The skin contact adhesive of this invention may be used, for example, in the transdermal drug delivery systems described in U.S. Pat. Nos. 4,840,796 and 4,951,657; and U.S. Patent Application Publications US2005/0048104 and US2007/0172518.

### Coating Composition

The copolymer composition described above may alternatively be used in a coating composition, e.g., for forming a coating on a substrate. The coating composition comprises: (a) the copolymer composition described above and (b) a coating additive. The coating additive may be selected from (b1) a water scavenger, (b2) a pigment, (b3) a diluent, (b4) a filler, (b5) a rust inhibitor, (b6) a plasticizer, (b7) a thickening agent, (b8) a pigment dispersant, (b9) a flow aid, (b10) a solvent, (b11) an adhesion promoter, (b12) a catalyst, (b13) an organic co-binder, (b14) a siloxane co-binder, (b15) a matting agent, (b16) a leveling agent, (b17) a wax, (b18) a texturizing additive, (b19) an anti-scratching additive, (b20) a gloss modifying additive, (b21) a stabilizer, and (b22) a crosslinker, or a combination of two or more of (b1), (b2), (b3), (b4), (b5), (b6), (b7), (b8), (b9), (b10), (b11), (b12)(b13), (b14), (b15), (b16), (b17), (b18), (b19), (b20), (b21) and (b22). Suitable fillers include silica and titanium dioxide, or zirconium dioxide. Suitable adhesion promoters include alkoxysilanes such as 3-glycidoxypropyltrimethoxysilane. Suitable solvents are as described above in the method for making the copolymer. Examples of suitable (b2) pigments, (b3) diluents, (b4) fillers, (b5) rust inhibitors, (b6) plasticizers, (b7) thickening agents, (b8) pigment dispersants, (b9) flow aids, (b10) solvents, and (b11) adhesion promoters are disclosed in U.S. Patent Application Publication Number 2015/0031797 and PCT Publications WO2015/097064, WO2015/100258, and WO2016/126362. The catalyst used in the coating composition as starting material (b12) may be the same as described as starting material e) described above and may be present in the coating composition in an amount of 0.01% to 5.00% by weight based on combined weights of all starting materials used to make the coating composition. Starting material (b13) is an organic co-binder such as a polyol, polyamine, or polyisocyanate; which can be added to the coating composition in an amount of 0 to 99% based on combined weights of all starting materials used to make the coating composition. Starting material (b14) is a siloxane co-binder that may be added in an amount of 0 to 99%, based on combined weights of all starting materials in the coating composition. Starting material (b15) is a matting agent that can be 0 to 30% based on combined weights of all starting materials in the coating composition. Starting material (b16) is a leveling agent, which can be present in an amount of 0 to 10% of the coating composition. Starting material (b17) is a wax, which can comprise 0 to 20% of the coating composition described herein. Starting material (b18) is a texturing additives that can be added to the coating composition in an amount of 0 to 20%. Starting materials (b19), (b20), (b21), and (b22) combined, can be 0 to 15%, all based on the total amount of all starting materials in the coating composition.

The coating composition may be hardened to form a coating such as a primer or a top coat on a substrate. The substrate can be a metal, glass, wood, painted layer, plastic foil, a fiber and/or textile, or leather. The coating composition can be applied to the substrate, e.g., fiber and/or textile during making the fibers or textiles, or later such as during laundering textiles. After application, solvent (if any) can be removed from the coating composition for example by drying the coating composition at ambient or elevated temperature. The amount of treatment composition applied to the substrate, e.g., fibers and textiles is typically sufficient to provide 0.1 to 15 weight percent of the composition on the substrate, based on the dry weight of the substrate, alternatively in an amount of 0.2 to 5 weight percent based on the dry weight of the substrate.

Fibers and textiles that can be treated with the treatment composition include natural fibers such as cotton, silk, linen, and wool; regenerated fibers such as rayon and acetate; synthetic fibers such as polyesters, polyamides, polyacrylonitriles, polyethylenes, and polypropylenes; combinations, and blends thereof. The form of the fibers can include threads, filaments, tows, yarns, woven fabrics, knitted materials, non-woven materials, paper, and carpet. For purposes of this application, additional substrates can be treated with the treatment composition, including leather. Without wishing to be bound by theory, it is thought that textiles treated with the silicone block copolymer have a feel on hand comparable to conventional hydrophobic silicone, but do not significantly impact negatively on the hydrophilicity of the textile. Without wishing to be bound by theory, it is thought that a coating formed from the coating composition described above may have one or more benefits of high gloss, flexibility, hardness, scratch resistance, and resistance to weathering, resistance to ultra-violet radiation exposure, or two or more thereof.

### EXAMPLES

Some embodiments of the invention will now be described in detail in the examples below. Reference Examples are not prior art unless so indicated.

TABLE A
Abbreviations

| Abbreviation | Meaning |
|---|---|
| AA | Allyl amine from Aldrich |
| AOH | Allyl alcohol |
| BD | 1,4-butanediol |
| DA | Diallyl amine from TCI |
| DMA | N,N-dimethylacetamide |
| EtAc | Ethyl acetate, from Sigma-Aldrich. Anhydrous |
| | for polymerization reaction. HPLC grade for |
| | dissolving and processing copolymers. |
| HDI | Hexamethylene diisocyanate from Acros |
| IPDI | Isophorone diisocyanate from Alfa Aesar |
| TDI | Toluene Diisocyanate |
| THF | Tetrahydrofuran |
| C16, | Carbinol terminated polydimethylsiloxane |
| | having MW of 920 to 924 from Gelest, Product |
| | DMS-C16 |
| C21 | Carbinol terminated polydimethylsiloxane |
| | having MW of 4330 to 4680 from Gelest, |
| | Product DMS-C21 |
| C23 | Carbinol terminated polydimethylsiloxane |
| | having MW of 12000 from Gelest, Product |
| | DMS-C23 |
| C62 | Carbinol terminated polydimethylsiloxane |
| | with a molecular weight MW of 1670 from |
| | Dow Corning |
| FTIR | Fourier Transform Infra-Red |
| GPC | Gel Permeation Chromatography |
| NMR | Nuclear Magnetic Resonance |
| Ml | Milliliters |
| ° C. | Degrees Celsius |
| Mg | Milligrams |
| Mn | Number average molecular weight determined |
| | by NMR |
| NMR | Nuclear magnetic resonance |
| XX-3035 | Trimethylsiloxy terminated dimethylsiloxane- |
| | methylmercaptopropylsiloxane copolymer with |
| | a SH content of 0.35 mol/100 g, made in the |
| | lab and analyzed by ²⁹Si and ¹H NMR. |
| SMS 142 | Trimethylsiloxy terminated dimethylsiloxane- |
| | methylmercaptopropylsiloxane copolymer with |
| | a SH content of 0.16 mol/100 g, purchased |
| | from Gelest, Inc. |
| SMS 042 | Trimethylsiloxy terminated dimethylsiloxane- |
| | methylmercaptopropylsiloxane copolymer with |
| | a SH content of 0.05 mol/100 g, purchased |
| | from Gelest, Inc. |
| 26298-125 | Trimethylsiloxy terminated dimethylsiloxane- |
| SH | methylmercaptopropylsiloxane copolymer with |
| crosslinker | a SH content of 0.16 mol/100 g, made in the |
| | lab and analyzed by ²⁹Si and ¹H NMR. |
| Darocur | 2-Hydroxy-2-methyl-1-phenyl-propan-1-one |
| 1173 | |
| N/A | Not available (not measured) |

### Reference Example—General Procedure for Preparing Copolymers

A 500 ml 4 neck flask was placed into a temperature controlled heating block and fitted with mechanical stirrer, thermometer, dropping funnel and reflux condenser.

1) The flask was charged with an a) isocyanate compound and a b) polyorganosiloxane, which were mixed to form a mixture.

2) The mixture was stirred and heated at 70° C. for a period of time after which solvent was added and the reaction cooled to below 40° C.

3) The residual isocyanate compound was added.

4) An c) chain extender and (optionally d) cross linker together with additional solvent were charged to the dropping funnel and added drop-wise to the mixture in the flask, which was then heated at 70° C. for a period of time.

5) The mixture in the flask was cooled to room temperature and poured into 2 liters (L) of deionized water. The precipitated copolymer was washed with additional water, collected and placed into a vacuum oven and dried at 75° C. and 50 millibars (mbar) for 24 hours.

Samples were prepared according to this procedure using starting materials and conditions shown in Table 1.

TABLE 1
Copolymer Preparation

| | | | | | a) | c) | |
|---|---|---|---|---|---|---|---|
| | a) | b) | Time for | | Isocyanate | chain | d) |
| | isocyanate | polyorgano- | heating | solvent in | compound | extender in | cross linke [symbol] |
| Example | compound | siloxane | in step 2) | step 2) | in step 3) | step 4) | in step 4) |
| 1 DMS-C16-PU32 | 7.8 g TDI | 47.3 g C16 | 4 hours | 120 ml THF | 2.7 g TDI | 1.4 g | No step 4) |
| copolymer (20% | | | | | | butane-1,4- | |
| Hard Seament | | | | | | diol | |
| Content) | | | | | | | |
| 2 DMS-C16-PU33 | 7.8 g TDI | 47.3 g C16 | 4 hours | 60 ml THF | 2.7 g TDI | 1.4 g | No step 4) |
| copolymer (20% | | | | 60 ml DMA | | butane-1,4- | |
| Hard Seament | | | | | | diol | |
| Content) | | | | | | | |
| 3 DMS-C16-PU34 | 6.6 g TDI | 40.0 g C16 | 4 hours | 60 ml THF | 1.3 g TDI | 0.7 g | No step 4) |
| copolymer (17.5% | | | | 60 ml DMA | | butane-1,4- | |
| Hard Seament | | | | | | diol | |
| Content) | | | | | | | |
| 4 DMS-C16-PU39 | 5.1 g TDI | 31.1 g C16 | 4 hours | 40 ml THF | 1.0 g TDI | 0.3 g | 0.2 g 1,1,1 [symbol] |
| copolymer (17.5% | | | | 40 ml DMA | | butane-1,4- | Trishydrox [symbol] |
| Hard Seament | | | | | | diol | methyletha |
| Content) | | | | | | | |
| 5 DMS-C16-PU40 | 5.1 g TDI | 31.1 g C16 | 4 hours | 40 ml THF | 1.0 g TDI | 0.4 g | 0.15 g 1,1, [symbol] |
| copolymer (17.5% | | | | 40 ml DMA | | butane-1,4- | Trishydrox [symbol] |
| Hard Seament | | | | | | diol | methyletha |
| Content) | | | | | | | |
| 6 PU-PDMS-BD | 13.9 g TDI | 31.8 g C16 | 4 hours | 60 ml THF | No step 3) | 4.5 g | No step 4) |
| copolymer 36.5% | | | | 60 ml DMA | | butane-1,4- | |
| hard Seament | | | | | | diol | |
| content. DMS- | | | | | | | |
| C21-PU 23 | | | | | | | |

[symbol]

indicates data missing or illegible when filed

Table 2 shows the NMR results and GPC determined molecular weights (Mw), where available, of the examples in Table 1. ¹H-NMR analysis (in ppm, solvent CDCl₃) analysis was performed. GPC conditions were: THF (1.0 ml/min) at 35° C.; column: Polymer Laboratories PLgel 5 μm Mixed-C columns; detector: Waters 2410 differential refractometer.

TABLE 2
Copolymer Characterization Results

| | Molecular | |
|---|---|---|
| | Weight, | |
| Example | Mw(g/mol) | ¹H-NMR analysis |
| 1 | | Aromatic (7.75, 7.06, 6.58, 6.36), |
| | | NH (4.65), —CH₂OOC (4.22-4.15), |
| | | —CH₂OOC (4.12-4.07), —CH₂—OH |
| | | (3.60-3.56), —CH₃ (2.17), —CH₂— |
| | | (1.72-1.63, 1.59-1.51), —CH₂—Si |
| | | (0.58-0.53), Si—CH₃ (0.09-0.03). |
| 2 | 61200 | Aromatic (7.75, 7.06, 6.58, 6.36), |
| | | NH (4.65), —CH₂OOC (4.22-4.15), |
| | | —CH₂OOC (4.12-4.07), —CH₂—OH |
| | | (3.60-3.56), —CH₃ (2.17), —CH₂— |
| | | (1.72-1.63, 1.59-1.51), —CH₂—Si |
| | | (0.58-0.53), Si—CH₃ (0.09-0.03). |
| 3 | | Aromatic (7.75, 7.06, 6.58, 6.36), |
| | | NH (4.65), —CH₂OOC (4.22-4.15), |
| | | —CH₂OOC (4.12-4.07), —CH₂—OH |
| | | (3.60-3.56), —CH₃ (2.17), —CH₂- |
| | | (1.72-1.63, 1.59-1.51), —CH₂—Si |
| | | (0.58-0.53), Si—CH₃ (0.09-0.03). |
| 4 | | Aromatic (7.75, 7.06, 6.58, 6.36), |
| | | NH (4.65), —CH₂OOC (4.22-4.15), |
| | | —CH₂OOC (4.12-4.07), —CH₂—OH |
| | | (3.60-3.56), —CH₃ (2.17), —CH₂— |
| | | (1.72-1.63, 1.59-1.51), —CH₂—Si |
| | | (0.58-0.53), Si—CH₃ (0.09-0.03). |
| 5 | | Aromatic (7.75, 7.06, 6.58, 6.36), |
| | | NH (4.65), —CH₂OOC (4.22-4.15), |
| | | —CH₂OOC (4.12-4.07), —CH₂—OH |
| | | (3.60-3.56), —CH₃ (2.17), —CH₂— |
| | | (1.72-1.63, 1.59-1.51), —CH₂—Si |
| | | (0.58-0.53), Si—CH₃ (0.09-0.03). |
| 6 | 34700 | Aromatic (7.73, 7.20, 7.03, 6.48), |
| | | NH (4.65), —CH₂OOC (4.19), —CH₂OOC |
| | | (4.08-4.06), —CH₂—OH (3.60-3.56), |
| | | —CH₃ (2.16), —CH₂— (1.76-1.64), |
| | | —CH₂—Si (0.57-0.52), Si—CH₃ |
| | | (0.08-0.03). |

### Example 1. Formulation of DMS-C16-PU32 Copolymer (20% Hard Segment Content) for Making Adhesion Test Samples

5 gm of the copolymer designated DMS-C16-PU32 was weighed into a vial, and 5 grams (gm) of Ethyl Acetate was added. This material was then mixed until all the copolymer was dissolved in Ethyl Acetate and a clear solution was obtained. This mixture was then coated onto a Mylar sheet using a 20 mil drawdown bar. A standard pressure sensitive adhesive (PSA) procedure was followed to obtain the laminate. Since all the copolymer was a 50% solution in Ethyl acetate, the solvent in the coating was evaporated at room temperature in a fume hood first and then the coating was dried at 95° C. for 5 minutes in a vented oven. The laminate was further covered with a LDPE release liner and left overnight at room temperature. Each sample was then tested for adhesion, peel release and cohesive strength.

For the release measurement, the release liner was secured in the bottom clamp and the adhesive coated polyurethane laminate was secured in the top clamp. The clamps were pulled apart at 10 mm/s for 130 mm. The value reported for each test is the average force (N)/in to pull the release liner from the adhesive coated polyurethane laminate. The data from the first 20 mm and the last 10 mm were discarded and the data from the remaining 100 mm was averaged. One to three replicates were tested to generate the report value with the final measurement in Newtons per (linear) inch (N/in). The final reported value is the average of the 1 to 3 test strips (1 inch=˜25 mm).

For the adhesion measurement, the release liner was removed from the laminate, which was adhered to the frosted side of a 1.5 in ×9 in (3.8 cm×23 cm) strip of polycarbonate. With the use of a 5 pound (Ib) rubber coated roller, the laminate was applied to the polycarbonate with one stroke forward and one stroke back at a rate of 1 in/sec (2.5 cm/sec). The sample was allowed to remain in contact with the polycarbonate for 30 minutes. During the test, the polycarbonate was secured in the bottom clamp while the laminate was secured in the top clamp. As in the release test, the clamps were pulled apart at 10 mm/s for 130 mm. The force to pull the laminate (1 in wide) from the polycarbonate was averaged over 100 mm (excluding the first 20 mm and last 10 mm of the 130 mm pull) with the final measurement in Newtons per (linear) inch (N/in). The final reported value is the average of 1 to 3 tests.

Percent cohesive failure was evaluated by visually estimating the amount of adhesive remaining on the polycarbonate after testing for adhesion. When possible, a distinction was made between cohesively failing through the adhesive (true cohesive failure) versus transferring from the polyurethane substrate to the polycarbonate (adhesive failure at the substrate). Any adhesive remaining on the polycarbonate was referred to as indicating cohesive failure. These adhesion test results are included in Table 3.

### Example 2. Formulation of DMS-C16-PU33 Copolymer (20% Hard Segment Content)

The copolymer designated DMS-C16-PU32 (5 gm) was weighed into a glass vial and 5 gm of Ethyl Acetate was added. The contents of the vial were then mixed on a vortex mixer until all the copolymer was dissolved in Ethyl Acetate and a clear solution was obtained. Laminate articles were prepared and evaluated for adhesion, peel release, and cohesive strength as in Example 1.

### Example 3. Formulation of DMS-C16-PU34 Copolymer (17.5% Hard Content)

5 gm of DMS-C16-PU34 was weighed into glass vial and 5 gm of Ethyl Acetate was added. This material was then mixed on a vortex mixer until all the polymer was dissolved in Ethyl Acetate and a clear solution was obtained. Laminate articles were prepared and tested as in Example 1.

### Example 4. Formulation of DMS-C16-PU39 Copolymer (17.5% Hard Content)

5 gm of DMS-C16-PU39 was weighed into a glass vial and 5 gm of Ethyl Acetate was added. The material was then mixed on a vortex mixer until all the polymer was dissolved in Ethyl Acetate and a clear solution was obtained. Laminate articles were prepared and tested as in Example 1.

### Example 5. Formulation of DMS-C16-PU40 Copolymer (17.5% Hard Content)

5 gm of DMS-C16-PU40 was weighed into a glass vial and 5 gm of Ethyl Acetate was added. The material was then mixed on a vortex mixer until all the polymer was dissolved in Ethyl Acetate and a clear solution was obtained. Laminate articles were prepared and tested as in Example 1.

### Example 6. Blending Formulation of DMS-C16-PU23 (36.5% Hard Content) and DMS-C16-PU33 (20% Hard Content)

3 gm of 50% DMS-C16-PU23 solution in Ethyl Acetate and 1.5 gm of 50% DMS-C16-PU33 solution in Ethyl Acetate, prepared as described above, were weighed into a glass vial. Each solution was then mixed at on a vortex mixer until a clear solution was obtained. The solutions were then mixed at different ratios as seen in Table 4. Laminate articles were prepared and tested as in Example 1.

A series of formulations were prepared using the same copolymers but changing the ratio of hard to soft content in the formulations. All the formulations were transferred into the laminates and were tested for adhesion, peel release and cohesive strength following the procedure in Example 1.

TABLE 3
Results for Examples 1-5

| | Peel | Adhesion | Cohesive | Anchorage | Cohesive |
|---|---|---|---|---|---|
| | Force | to PC | (adh) | to PU | (anc) |
| Copolymer | (N/25 mm) | (N/25 mm) | % | (N/25 mm) | % |
| DMS-C16- PU 32 | 0.361 | 14.455 | 100 | 16.736 | 100 |
| Copolymer 20% HC | | | | | |
| DMS-C16- PU 33 | 0.107 | >30 | 100 | 25.401 | 100 |
| Copolymer 20% HC | | | | | |
| DMS-C16- PU 34 | 0.636 | 9.681 | 100 | 13.234 | 100 |
| Copolymer 17.5% | | | | | |
| HC | | | | | |
| DMS-C16- PU 39 | 1.212 | 7.583 | 100 | 9.74 | 100 |
| Copolymer 17.5% | | | | | |
| HC | | | | | |
| DMS-C16- PU 40 | 0.275 | 30.064 | 100 | 26.986 | 100 |
| Copolymer 17.5% | | | | | |
| HC | | | | | |

TABLE 4
Results for Example 6

| | | Cured | | | |
|---|---|---|---|---|---|
| | Shim | Film | Peel | Adhesion | Cohesive |
| Copolymers Blend | Thickness | Thickness | Force | (N/Inch) | Failure |
| ratio | (mil) | (mil) | (N/Inch) | PC | (adh)% |
| 16.7% of 36.5% | 20 | 7-7.3 | 0.058 | 21.708 | 50 |
| HC + 84.3% of | | | | | |
| 20% HC | | | | | |
| 8.3% of 36.5% | 6 | 4 | 0.044 | 22.760 | 50 |
| HC + 91.7% of | | | | | |
| 20% HC | | | | | |
| 16.7% of 36.5% | 6 | 4-4.3 | 0.056 | 13.826 | 40 |
| HC + 84.3% of | | | | | |
| 20% HC | | | | | |
| 30% of 36.5% | 6 | 3.3-3.7 | 0.102 | 12.678 | 35-40 |
| HC + 70% of | | | | | |
| 20% HC | | | | | |
| 50% of 36.5% | 6 | 3 | 0.003 | 0.646 | No CF |
| HC + 50% of | | | | | |
| 20% HC | | | | | |
| 40% of 36.5% | 16 | 7-7.3 | 0.011 | 2.971 | No CF |
| HC + 60% of | | | | | |
| 20% HC | | | | | |
| 40% of 36.5% | 16 | 9.3 | 0.129 | 3.557 | 2-5% Res. |
| HC + 60% of | | | | | on PC |
| 20% HC | | | | | |
| 35% of 36.5% | 8 | 5-5.3 | 0.023 | 12.814 | 10-20 |
| HC + 65% of | | | | | |
| 20% HC | | | | | |
| 37% of 36.5% | 8 | 4 | 0.029 | 2.983 | No CF |
| HC + 63% of | | | | | |
| 20% HC | | | | | |

### Example 7—Copolymer Synthesis

Samples of siloxane-urethane-urea copolymers were prepared by combining a carbinol functional polydimethylsiloxane (C62) with isophorone diisocyanate in amounts shown below in Table 5. Polyethylene glycols (PEG600 and PEG1500) with different molecular weights were optionally added. The starting materials described above were combined in a flask with methyl isobutyl ketone solvent and heated to 60° C. with stirring. A catalyst (bismuth neodecanoate) was then added. The temperature was increased to 80 C, and the contents of the flask were heated at 80 C for 2 hours. The isocyanate content was determined via titration following DIN EN ISO 11909.

TABLE 5
Raw materials and amounts to make siloxane-
urethane-urea copolymers in Example 7.

| Sample no. | 8-1 | 8-2 | 8-3 | 8-4 | 8-5 | 8-6 | 8-7 |
|---|---|---|---|---|---|---|---|
| n_{OH}(PEG)/ | 14.65/ | 85.35/ | 50/ | 85.35/ | 14.65/ | 50/ | 50/ |
| n_{OH}(C62) | 85.35 | 14.65 | 50 | 14.65 | 85.35 | 50 | 50 |
| Ratio n_{NCO}/ | 1.7 | 1.4 | 1.55 | 1.4 | 1.4 | 1.55 | 1.55 |
| n_{OH} | | | | | | | |
| PEG-type | 600 | 1500 | 600 | 600 | 600 | 1500 | 1500 |
| Diisocyanate/g | 15.3 | 12.2 | 17.5 | 21.1 | 13.1 | 13.2 | 13.2 |
| (IPDI) | | | | | | | |
| C62/g | 51.2 | 8.5 | 37.6 | 14.8 | 53.3 | 28.5 | 28.5 |
| Organic diol/g | 3.5 | 0.0 | 14.9 | 34.1 | 3.6 | 0.0 | 0.0 |
| (PEG 600) | | | | | | | |
| Organic diol/g | 0.0 | 49.3 | 0.0 | 0.0 | 0.0 | 28.2 | 28.2 |
| (PEG 1500) | | | | | | | |
| Bi catalyst | 0.2 | 0.2 | 0.2 | 0.2 | 0.2 | 0.2 | 0.2 |
| solution 10%/g | | | | | | | |
| (Bi neodecanoate | | | | | | | |
| in PPG400) | | | | | | | |
| Methyl ethyl | 30.0 | 30.0 | 30.0 | 30.0 | 30.0 | 30.0 | 30.0 |
| ketone/g | | | | | | | |

| Sample no. | 8-8 | 8-9 | 8-10 | 8-11 | 8-12 | 8-13 | 8-14 |
|---|---|---|---|---|---|---|---|
| n_{OH}(PEG)/ | 14.65/ | 85.35/ | 50/ | 85.35/ | 14.65/ | 50/ | 50/ |
| n_{OH}(C62) | 85.35 | 14.65 | 50 | 14.65 | 85.35 | 50 | 50 |
| Ratio n_{NCO}/ | 1.4 | 1.7 | 1.55 | 1.7 | 1.7 | 1.55 | 1.55 |
| n_{OH} | | | | | | | |
| PEG-type | 1500 | 1500 | 600 | 600 | 1500 | 1500 | 600 |
| Diisocyanate/g | 12.1 | 14.3 | 17.5 | 24.1 | 14.2 | 13.2 | 17.5 |
| (IPDI) | | | | | | | |
| C62/g | 49.5 | 8.2 | 37.6 | 13.9 | 47.7 | 28.5 | 37.6 |
| Organic diol/g | 0 | 0 | 14.9 | 32.0 | 0 | 0 | 14.9 |
| (PEG 600) | | | | | | | |
| Organic diol/g | 8.4 | 47.5 | 0 | 0 | 8.1 | 28.2 | 0 |
| (PEG 1500) | | | | | | | |
| Bi catalyst | 0.2 | 0.2 | 0.2 | 0.2 | 0.2 | 0.2 | 0.2 |
| solution 10%/g | | | | | | | |
| (Bi neodecanoate | | | | | | | |
| in PPG400) | | | | | | | |
| Methyl ethyl | 30.0 | 30.0 | 30.0 | 30.0 | 30.0 | 30.0 | 30.0 |
| ketone/g | | | | | | | |

| Sample no. | 8-15 | 8-16 |
|---|---|---|
| n_{OH}(PEG)/n_{OH}(siloxane diol) | 100/0 | 100/0 |
| Ratio n_{NCO}/n_{OH} | 1.55 | 1.55 |
| PEG-type | 600 | 1500 |
| Diisocyanate (IPDI)/g | 25.9 | 13.3 |
| Siloxane diol A/g | 0 | 0 |
| Organic diol (PEG 600)/g | 44.1 | 0 |
| Organic diol (PEG 1500)/g | 0 | 56.7 |
| Bi catalyst solution 10%/g | 0.2 | 0.2 |
| (Bi neodecanoate in PPG400) | | |
| Methyl ethyl ketone/g | 30.0 | 30.0 |

PPG 400 is polypropylene glycol. All the organic-siloxane copolymers prepared in Example 7 were clear and homogeneous solutions in MEK. Residual NCO levels were measured in accordance with theoretical calculated values.

### Example 9—Coatings

The copolymers prepared in Example 7 were formulated in coating compositions that were then hardened to prepare coatings. The coating compositions contained the starting materials in amounts shown below in Table 6. The coating compositions were prepared by mechanically blending the starting materials.

TABLE 6
Coating Compositions

| Sample no. | A | B | C | D | E |
|---|---|---|---|---|---|
| Copolymer 8-3/g | 11.0 | 11.0 | 0.0 | 0.0 | 0.0 |
| Copolymer 8-5/g | 0.0 | 0.0 | 11.0 | 0.0 | 0.0 |
| Copolymer 8-15/g | 0.0 | 0.0 | 0.0 | 11.0 | 0.0 |
| Organic polyol/g | 4.5 | 0.0 | 2.7 | 6.6 | 3.4 |
| (Desmophen A 870 BA) | | | | | |
| Organic polyol/g | 0.0 | 0.2 | 0.0 | 0.0 | 0.0 |
| (Glycerol) | | | | | |
| Bi catalyst solution | 0.2 | 0.2 | 0.2 | 0.2 | 0.2 |
| 10%/g (Bi neodecanoate | | | | | |
| in PPG 400) | | | | | |
| Water scavenger/g | 1.0 | 1.0 | 1.0 | 1.0 | 1.0 |
| (Trimethyl orthoformate) | | | | | |
| Butyl acetate/g | 0.0 | 0.1 | 0.0 | 0.0 | 0.0 |

The coating compositions in Table 6 were coated on Aluminum Q-Panels Q36. The film thickness was 100 micrometers (μm). The compositions were cured by exposure to ambient conditions for 7 days before testing. Each coating on the substrate had a smooth and homogeneous surface.

A comparative sample (“0”) was prepared by a mechanically blending the compositions shown in Table 7, below. Additionally, a full organic coating composition was prepared as reference (“00”).

TABLE 7
Comparative Coating Compositions

| Sample no. | 0 | 00 |
|---|---|---|
| n_{OH}(Polyol)/n_{OH}(siloxane | 59/41 | 100/0 |
| diol) | | |
| Isocyanate pre-polymer/g | 11.0 | 10.0 |
| Organic polyol/g | 27.2 | 11.9 |
| Siloxane diol A/g | 21.6 | 0.0 |
| Bi catalyst solution | 0.2 | 0.2 |
| 10%/g (Bi neodecanoate | | |
| in PPG 400) | | |
| Water scavenger/g | 1.0 | 1.0 |
| (Trimethyl orthoformate) | | |
| Butyl acetate/g | 7.0 | 7.0 |

The compositions in Table 7 were applied to substrates and cured as described above for the compositions in Table 6. Coating composition “00” produced a clear and homogeneous film. Coating “0” generated a nonhomogeneous liquid composition, and after application, a film surface with a lot of pinholes and craters. This approach shows that the use of compositions containing the polyurethane-polyorganosiloxane copolymer allows improved coating formation, and the silicone character to be sensitively enhanced in these compositions.

Table 8 shows durability test results of coatings from the compositions in Table 6.

TABLE 8
Test results of coatings in Table 6.

| Sample | A | B | C | D | E |
|---|---|---|---|---|---|
| Pendulum hardness König | 11 | 31 | 18 | 9 | 34 |
| Hardness Clemen | 550 | 500 | 350 | 800 | 700 |
| Water Contact angle (°) | 101 | 108 | 107 | 80 | 82 |
| at t + 30″ | | | | | |

Table 8 shows that water repellency for the coatings made with the organic-siloxane copolymer described herein have sensitively higher than organic systems. As siloxane units are chemically bonded, it is thought that there will not be any wash-off during aging of the layer. König and Clemen values show a higher flexibility of the layer, with lower risk of cracking during handling of the paint, and during aging of the coating. The Clemen test is a measure of the coating hardness using a tungsten needle applied on the coating at increasing pressure. A raise indicates the failure in the electrical intensity indicating a failure of the coating. The method is ISO 1518. The water contact angle is measured with a drop of water deposited on the coating and measured using a camera equipped with a digital goniometer. The angle at TO and T30 are recorded. This is following DIN norm: DIN 55660-2.

Without wishing to be bound by theory, it is thought that the coatings prepared as described above will exhibit one or more of the following benefits: improved compatibility between polyurethane and silicones, improved weathering resistance, hydrophobicity, hydrolytic stability, radiation resistance, thermal resistance, corrosion resistance, surface smoothness and gloss, scratch resistance, lower viscosity at similar solid content (impacting volatile organic content, VOC), and reduced friction, and that these benefits will be more durable if the siloxane-based material is chemically bonded to the urethane backbone (as in the copolymers described herein) instead of mechanically dispersing siloxane in urethane in the coating layer.

1. A copolymer composition comprising: two or more starting materials, where
(I) at least one of the starting materials is copolymer (A) or copolymer (B), where copolymer (A) is a siloxane-urethane-urea copolymer of unit formula 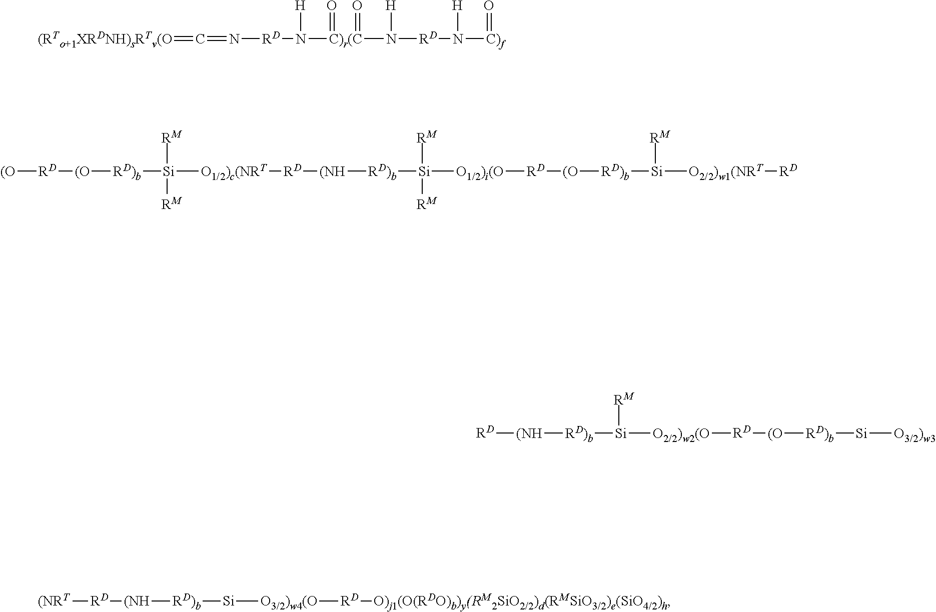
where
each R^{D} is independently a divalent hydrocarbon group a divalent halogenated hydrocarbon group;
each R^{M} is independently a monovalent hydrocarbon group or a monovalent halogenated hydrocarbon group;
each R^{T} is independently hydrogen or a hydrocarbon group;
each subscript b is independently 0 to 1,000,000;
subscript c is 0 to 200,000, subscript i is 0 to 200,000, subscript w1 is 0 to 200,000, subscript w2 is 0 to 200,000, subscript w3 is 0 to 200,000, subscript w4 is 0 to 200,000, and a quantity (c+i+w1+w2+w3+w4) is ≥1;
subscript d is 0 to 1,000,000;
subscript e is 0 to 1,000,000;
subscript f is 0 to 1,500,000;
subscript h is 0;
subscript j1 is ≥0;
each X is independently nitrogen, oxygen, or sulfur;
subscript o=0 when X is oxygen or sulfur, and subscript o=1 when X is nitrogen;
subscript r is 0 to 1,500,000, and the quantity f+r is ≥1;
subscript s is 0 to 200,000; and
subscript v is 0 to 200,000;
subscript y is ≥0;
and
copolymer (B) is a siloxane-urethane-urea copolymer of the following formulae 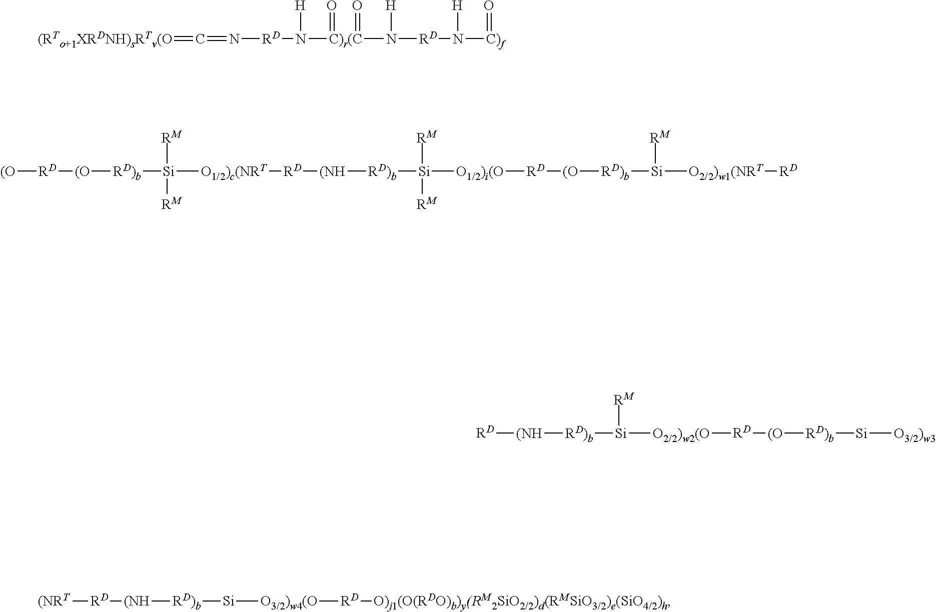
where all the symbols and subscripts are defined in A) and subscript j2 is >0, and if j1 is >0, then j2/j1 is ≥1.1; and
(II) optionally one or both of starting materials (C) and (D), where
(C) is an organic polyol; and
(D) is a reaction product of an organic polyisocyanate and an organic polyol.

2. The composition of claim 1, where the at least two starting materials are selected from:
(A) and (B);
(A) and (C);
(B) and (C);
(A) and (D);
(B) and (D);
(A), (B), and (C);
(A), (B), and (D);
(A), (C), and (D);
(B), (C), and (D); or
(A), (B), (C), and (D).

3. The composition of claim 1, further comprising (E) an organosiloxane polymer.

4. The composition of claim 1, where copolymer (A) is present, and copolymer (A) comprises unit formula (I): 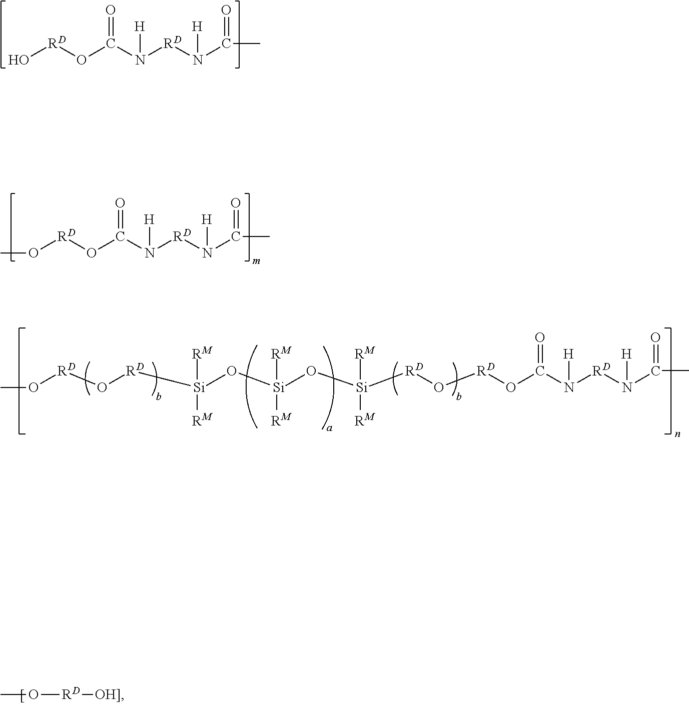
where each subscript a is independently 0 to 1,000,000; each subscript b is independently greater than or equal to 0; each subscript m is ≥0, and subscript n is greater than or equal to 1.

5. The composition of claim 1, where copolymer (B) is present, and copolymer (B) comprises unit formula (III): 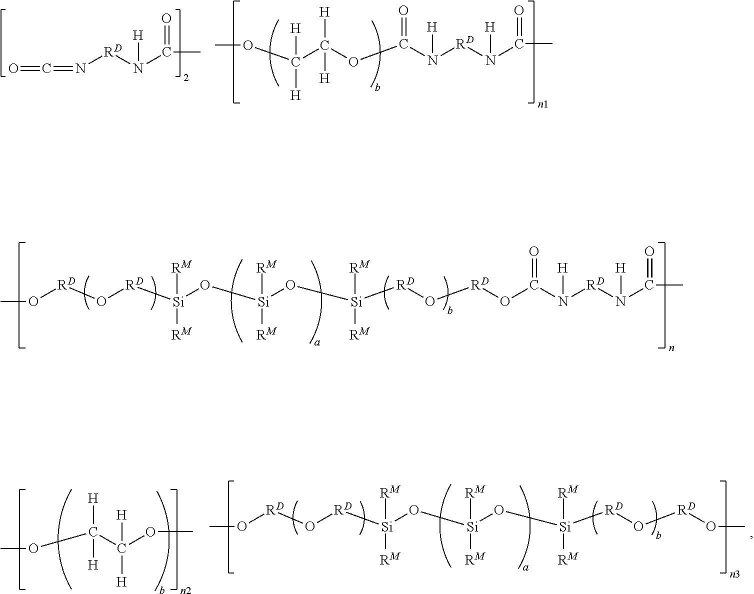
where subscript n is greater than or equal to 1, subscript n1 is greater than or equal to 0, subscripts n2 and n3 are 0 or 1, and a quantity (n2+n3)=1.

6. A skin contact adhesive composition comprising:
(I) a copolymer composition comprising: two or more starting materials, where
(a) at least one of the starting materials is copolymer (A) or copolymer (B), where copolymer (A) is a siloxane-urethane-urea copolymer of unit formula 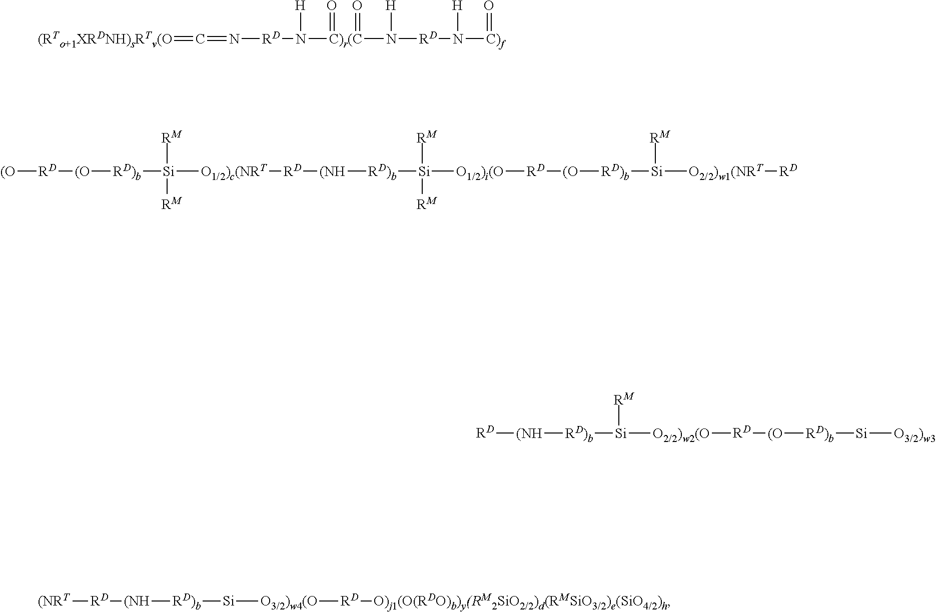
where
each R^{D} is independently a divalent hydrocarbon group a divalent halogenated hydrocarbon group;
each R^{M} is independently a monovalent hydrocarbon group or a monovalent halogenated hydrocarbon group;
each R^{T} is independently hydrogen or a hydrocarbon group;
each subscript b is independently 0 to 1,000,000;
subscript c is 0 to 200,000, subscript i is 0 to 200,000, subscript w1 is 0 to 200,000, subscript w2 is 0 to 200,000, subscript w3 is 0 to 200,000, subscript w4 is 0 to 200,000, and a quantity (c+i+w1+w2+w3+w4) is ≥1;
subscript d is 0 to 1,000,000;
subscript e is 0 to 1,000,000;
subscript f is 0 to 1,500,000;
subscript h is 0;
subscript j1 is 0;
each X is independently nitrogen, oxygen, or sulfur;
subscript o=0 when X is oxygen or sulfur, and subscript o=1 when X is nitrogen;
subscript r is 0 to 1,500,000, and the quantity f+r is ≥1;
subscript s is 0 to 200,000; and
subscript v is 0 to 200,000;
subscript y is ≥0;
and
copolymer (B) is a siloxane-urethane-urea copolymer of the following formulae 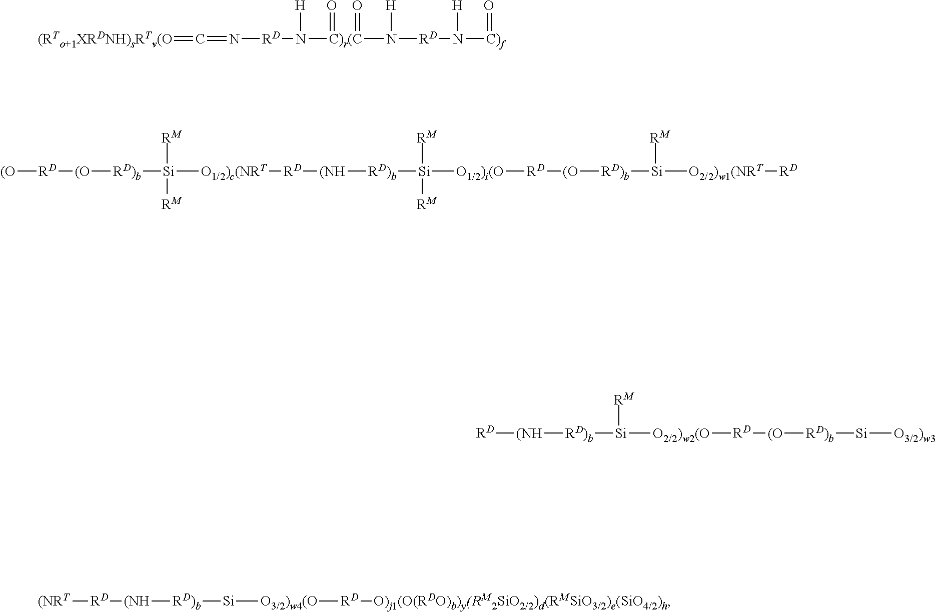
where all the symbols and subscripts are defined in A) and subscript j2 is ≥0, and if j1 is ≥0, then j2/j1 is ≥1.1; and
(b) optionally one or both of starting materials (C) and (D), where
(C) is an organic polyol; and
(D) is a reaction product of an organic polyisocyanate and an organic polyol;
and
(II) an excipient, and
optionally (III) an active ingredient.

7. The skin contact adhesive composition of claim 6, where the excipient is selected from (II-1) a stabilizer, (II-2) a binder, (II-3) a filler, (II-4) a solubilizer, (II-5) a skin penetration enhancer, (II-6) an adhesion promoter, (II-7) agent to improve moisture permeability, or a combination of two or more of (II-1), (II-2), (II-3), (II-4), (II-5), (II-6), and (II-7).

8. The skin contact adhesive composition of claim 6, where (III) the active ingredient is present, and the active ingredient is selected from drugs that act upon the central nervous system; drugs affecting renal function; drugs affecting cardiovascular function; drugs affecting gastrointestinal function; drugs for treatment of helminthiasis; antimicrobial agents such as silver, silver compounds, and/or chlorhexidine; nutrients; hormones; steroids; and drugs for treatment of dermatoses; non-steroidal anti-inflammatory drugs such as salicylates e.g., acetylsalicylic acid; propionic acid derivatives e.g., (RS)-2-(4-(2-Methylpropyl)phenyl)propanoic acid (ibuprofen); acetic acid derivatives e.g., 2-{1-[(4-Chlorophenyl)carbonyl]-5-methoxy-2-methyl-1H-indol-3-yl}acetic acid (indomethacin), enolic acid derivatives; anthranilic acid derivatives, COX-2 inhibitors e.g., N-(4-hydroxyphenyl)ethanamide N-(4-hydroxyphenyl)acetamide (acetaminophen), and sulfonanilides; local anesthetics such local anesthetics containing an ester group e.g., ethyl 4-aminobenzoate (benzocaine); local anesthetics containing an amide group e.g., 2-(diethylamino)-N-(2,6-dimethylphenyl)acetamide (lidocaine); and naturally derived local anesthetics e.g., (1R,2S,5R)-2-Isopropyl-5-methylcyclohexanol (menthol).

9. The skin contact adhesive composition of claim 8, where the active ingredient is selected from antimicrobial agents, non-steroidal anti-inflammatory drugs, and local anesthetics.

10. (canceled)

11. (canceled)

12. A coating composition comprising:
(a) two or more starting materials, where
(I) at least one of the starting materials is copolymer (A) or copolymer (B), where copolymer (A) is a siloxane-urethane-urea copolymer of unit formula 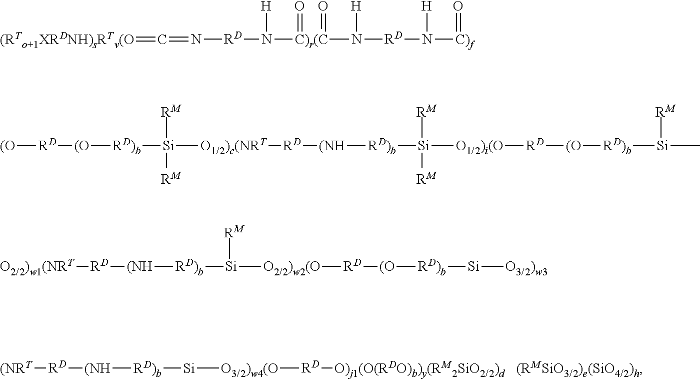
where
each R^{D} is independently a divalent hydrocarbon group a divalent halogenated hydrocarbon group;
each R^{M} is independently a monovalent hydrocarbon group or a monovalent halogenated hydrocarbon group;
each R^{T} is independently hydrogen or a hydrocarbon group;
each subscript b is independently 0 to 1,000,000;
subscript c is 0 to 200,000, subscript i is 0 to 200,000, subscript w1 is 0 to 200,000, subscript w2 is 0 to 200,000, subscript w3 is 0 to 200,000, subscript w4 is 0 to 200,000, and a quantity (c+i+w1+w2+w3+w4) is ≥1;
subscript d is 0 to 1,000,000;
subscript e is 0 to 1,000,000;
subscript f is 0 to 1,500,000;
subscript h is 0;
subscript i is 0;
each X is independently nitrogen, oxygen, or sulfur:
subscript o=0 when X is oxygen or sulfur, and subscript o=1 when X is nitrogen;
subscript r is 0 to 1,500,000, and the quantity f+r is ≥1;
subscript s is 0 to 200,000; and
subscript v is 0 to 200,000;
subscript y is 0;
and
copolymer (B) is a siloxane-urethane-urea copolymer of the following formulae 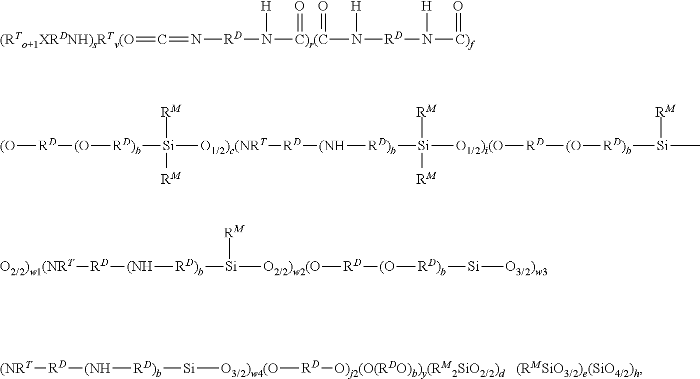
where all the symbols and subscripts are defined in A) and subscript j2 is ≥0, and if j1 is ≥0, then j2/j1 is ≥1.1; and
(II) optionally one or both of starting materials (C) and (D), where
(C) is an organic polyol; and
(D) is a reaction product of an organic polyisocyanate and an organic polyol; and
(b) a coating additive.

13. The coating composition of claim 12, where (b) the coating additive is selected from (b1) a water scavenger, (b2) a pigment, (b3) a diluent, (b4) a filler, (b5) a rust inhibitor, (b6) a plasticizer, (b7) a thickening agent, (b8) a pigment dispersant, (b9) a flow aid, (b10) a solvent, (b11) an adhesion promoter, (b12) a catalyst, (b13) an organic co-binder, (b14) a siloxane co-binder, (b15) a matting agent, (b16) a leveling agent, (b17) a wax, (b18) a texturizing additive, (b19) an anti-scratching additive, (b20) a gloss modifying additive, (b21) a stabilizer, and (b22) a crosslinker, or a combination of two or more of (b1), (b2), (b3), (b4), (b5), (b6), (b7), (b8), (b9), (b10), (b11), (b12)(b13), (b14), (b15), (b16), (b17), (b18), (b19), (b20), (b21) and (b22).

14. A method comprising:
i) applying the coating composition of claim 12 to a substrate, and
ii) hardening the composition to form a coating.

15. The method of claim 14, where the substrate comprises leather.
